SEQUENCE LISTING (8 sequences)

SEQ ID NO: 1 - DNA, 1323 bp, Homo sapiens
  sig_peptide (213)..(269)
aataggggaaaataagccagatggataaaggaagtgctggtcaccctggaggtgcactggtttggggaaggctcctggcccccacagccctcttcggagcctgagcccggctctcctcactcacctcaacccccaggcggcccctccacagggcccctctcctgcctggacggctctgctggtctccccgtcccctggagaagaacaaggccatgggtcggcccctgctgctgcccctactgcccttgctgctgccgccagcatttctgcagcctagtggctccacaggatctggtccaagctacctttatggggtcactcaaccaaaacacctctcagcctccatgggtggctctgtggaaatccccttctccttctattacccctgggagttagccacagctcccgacgtgagaatatcctggagacggggccacttccacaggcagtccttctacagcacaaggccgccttccattcacaaggattatgtgaaccggctctttctgaactggacagagggtcagaagagcggcttcctcaggatctccaacctgcagaagcaggaccagtctgtgtatttctgccgagttgagctggacacacggagctcagggaggcagcagtggcagtccatcgaggggaccaaactctccatcacccaggctgtcacgaccaccacccagaggcccagcagcatgactaccacctggaggctcagtagcacaaccaccacaaccggcctcagggtcacacagggcaaacgacgctcagactcttggcacataagtctggagactgctgtgggggtggcagtggctgtcactgtgctcggaatcatgattttgggactgatctgcctcctcaggtggaggagaaggaaaggtcagcagcggactaaagccacaaccccagccagggaacccttccaaaacacagaggagccatatgagaatatcaggaatgaaggacaaaatacagatcccaagctaaatcccaaggatgacggcatcgtctatgcttcccttgccctctccagctccacctcacccagagcacctcccagccaccgtcccctcaagagcccccagaacgagaccctgtactctgtcttaaaggcctaaccaatggacagccctctcaagactgaatggtgaggccaggtacagtggcgcacacctgtaatcccagctactctgaagcctgaggcagaatcaagtgagcccaggagttcagggccagctttgataatggagcgagatgccatctctagttaaaaatatatattaacaataaagtaacaaatttaaaaagataaaaaaa

SEQ ID NO: 2 - protein, 303 aa, Homo sapiens
  SIGNAL (1)..(19)
  mat_peptide (20)..(303)
MGRPLLLPLLPLLLPPAFLQPSGSTGSGPSYLYGVTQPKHLSASMGGSVEIPFSFYYPWELATAPDVRISWRRGHFHRQSFYSTRPPSIHKDYVNRLFLNWTEGQKSGFLRISNLQKQDQSVYFCRVELDTRSSGRQQWQSIEGTKLSITQAVTTTTQRPSSMTTTWRLSSTTTTTGLRVTQGKRRSDSWHISLETAVGVAVAVTVLGIMILGLICLLRWRRRKGQQRTKATTPAREPFQNTEEPYENIRNEGQNTDPKLNPKDDGIVYASLALSSSTSPRAPPSHRPLKSPQNETLYSVLKA

SEQ ID NO: 3 - DNA, 3632 bp, Homo sapiens
  sig_peptide (2497)..(2553)
agaagggaggtagtcgccctccgtcgtggcctggcgtggattccgagcgttggtgtctggcggtttccgaccgttggtgtctggcacgcgccaccccgatgtaccaggtaaagccctatcacggggtcggcgcccctctccgtgtggagcccacctgcatgtactggctccccaacatgcacggcaggagcggcggcccagcactcggcactggccacttgcagacaagaagacaagaaaatgatttgaggacagcttcaatcgcggtgtgaagaagaaagcaacaaaacgaccactgaaaacaatgccggtggcaaaacatccaaagaaagggtcccaagtggtacatcgtcatagctggaaacagtcagagccaccagccaatgatcttttcaatgctgcgaaagctgccaaaagtgacatgcagtgtggccatgaggtctgccggaagtgacttgttggtgttatctcctgagttaaaatgtgaagggatttttttttttcagattactgagagtcttcagttactagaggcggatttccctgactgaagaccatgttgcaggcccacagctgcctacagaaccgtcccaaaatatggcaaagaaacctattctgagcgatagggtctcaccatgttgcccaggctggtcttgaactcctggactcatcctaaagtgctggcctctcattccctgtctgtgcacacctcacggcaagggccagcctgtttcctcccggtcacctccaaatcttgctgcttttaattcaactcagaggcctagccagggttgagttctcacccacctgtgccgccctgccttgttacctggaagcacagccttggggactgagcaggccctcactgtcactttaagaagggaatcagccactttgtgctcaccacctctggggaaggtgtgagaggagagaaggaagtggctgtttggctgctgacaacatgaagacttcctgcgatgagaacagaggcacaggtgccggccctgcagcccccagaacccggactggagggggccatggggcgccggaccctggccctgccctgggtgctgctgaccctgcgtgtcactgcagggaccccggaggtgtgagtacaagttcggatggaggccaccgagctctcgtccttcaccatccgttgtgggttcctggagtctggctccatctccctggtgactgcccgctgggaaacccagagcagcatctctctcatcctggaaggctctggggccagcagcccctgcgccaacaccaccttctgctgcaagtttgcgtccttccctgagggctcctgggaggcctgtgggagcctcccgcccagctcagacccagggctctctgtcccgccgactcctgcccccattctgcgggcagacctggccgggatcttgggggtctcaggagtccttctctttgactgtggctacctccttcatctgctgtgccgacagaagcaccgccctgcccctaggctccagccatcccacaccagctcctaggcactgagagcacgagcatgggcacccagccaggcctcccaggctgctctccacgtcccttatgccactatcaacaccagctgctgcccagctactttggacacagctcacccccgacagggggccgtcctggtgggcatcactccccacccacaccgcacactggccccagggccctgctgcctgggcctccacatccatccctgcacatggcagctttgtctctgttgagaatggactctacgcttaggctgggcagaggcctccctgcactggtcctggcctcactcttttccctgacccttggggcccagggccatggagggacccttaggagttcaatgagagagaccatgaggccactgggctttccccttcccaggcctcctgggtgccacccccttacgttattcttgggcctctaataagtgtcccacaggtgcctggccaggcccacctgctgcagatgtggtctgtgtgtgtgcatgtgtgggtgtgtgtgggcacaggtgtgagtgtgtgagcaacagtaccccattccagtcgtttcctgctgtgactaagtcagcaacacagttcctctgacatgggccttggctgtgcttctttgggggtgaagagattggggaggaagtctccacccctgggaggcagaagccaggcatagcgcgctggctaggactccagtaccgtgaagggaggcagtgagagcagacatctgtgcctcattcctgatctcaaggggaaagcaagaacaagggaggcttcctcaggatctcgaacctgcggaaggaggaccagtctgtgtacttctgccaagtccagctggacatacagatcagggaggctgtcgtggcagtccatcaaggggacccacctcaccatcacccaggccctcaggcagcccctccacagggcccctctcctgcctggacagctctgctggtctccccgtcccctggagaagaacaaggccatgggtcggcccctgctgctgcccctgctgctcctgctgcagccgccagcatttctgcagcctggtggctccacaggatctggtccaagctacctttatggggtcactcaaccaaaacacctctcagcctccatgggtggctctgtggaaatccccttctccttctattacccctgggagttagccatagttcccaacgtgagaatatcctggagacggggccacttccacgggcagtccttctacagcacaaggccgccttccattcacaaggattatgtgaaccggctctttctgaactggacagagggtcaggagagcggcttcctcaggatctcaaacctgcggaaggaggaccagtctgtgtatttctgccgagtcgagctggacacccggagatcagggaggcagcagttgcagtccatcaaggggaccaaactcaccatcacccaggctgtcacaaccaccaccacctggaggcccagcagcacaaccaccatagccggcctcagggtcacagaaagcaaagggcactcagaatcatggcacctaagtctggacactgccatcagggttgcattggctgtcgctgtgctcaaaactgtcattttgggactgctgtgcctcctcctcctgtggtggaggagaaggaaaggtagcagggcgccaagcagtgacttctgaccaacagagtgtggggagaagggatgtgtattagccccggaggacgtgatgtgagacccgcttgtgagtcctccacactcgttccccattggcaagatacatggagagcaccctgaggacctttaaaaggcaaagccgcaaggcagaaggaggctgggtccctgaatcaccgactggaggagagttacctacaagagccttcatccaggagcatccacactgcaatgatataggaatgaggtctgaactccactgaattaaaccactggcatttgggggctgtttattatagcagtgcaaagagttcctttatcctccccaaggatggaaaaatacaatttattttgcttaccatacaccccttttctcctcgtccacattttccaatctgtatggtggctgtcttctatggcagaaggttttggggaataaatagcgtgaaatgctgctga

SEQ ID NO: 4 - protein, 227 aa, Homo sapiens
  SIGNAL (1)..(19)
  mat_peptide (20)..(227)
MGRPLLLPLLLLLQPPAFLQPGGSTGSGPSYLYGVTQPKHLSASMGGSVEIPFSFYYPWELAIVPNVRISWRRGHFHGQSFYSTRPPSIHKDYVNRLFLNWTEGQESGFLRISNLRKEDQSVYFCRVELDTRRSGRQQLQSIKGTKLTITQAVTTTTTWRPSSTTTIAGLRVTESKGHSESWHLSLDTAIRVALAVAVLKTVILGLLCLLLLWWRRRKGSRAPSSDF

SEQ ID NO: 5 - DNA, 1255 bp, Homo sapiens
  sig_peptide (175)..(240)
ggaggccggggcggggcgggcgcagccggcgctgagcttgcagggccgctcccctcacccgcccccttcgagtccccgggcttcgccccacccggcccgtgggggagtatctgtcctgccgccttcgcccacgccctgcactccgggaccgtccctgcgcgctctgggcgcaccatggcccgcggggctgcgctggcgctgctgctcttcggcctgctgggtgttctggtcgccgccccggatggtggtttcgatttatccgatgcccttcctgacaatgaaaacaagaaacccactgcaatccccaagaaacccagtgctggggatgactttgacttaggagatgctgttgttgatggagaaaatgacgacccacgaccaccgaacccacccaaaccgatgccaaatccaaaccccaaccaccctagttcctccggtagcttttcagatgctgaccttgcggatggcgtttcaggtggagaaggaaaaggaggcagtgatggtggaggcagccacaggaaagaaggggaagaggccgacgccccaggcgtgatccccgggattgtgggggctgtcgtggtcgccgtggctggagccatctctagcttcattgcttaccagaaaaagaagctatgcttcaaagaaaatgcagaacaaggggaggtggacatggagagccaccggaatgccaacgcagagccagctgttcagcgtactcttttagagaaatagaagattgtcggcagaaacagcccaggcgttggcagcagggttagaacagctgcctgaggctcctccctgaaggacacctgcctgagagcagagatggaggccttctgttcacggcggattctttgttttaatcttgcgatgtgctttgcttgttgctgggcggatgatgtttactaacgatgaattttacatccaaagggggataggcacttggacccccattctccaaggcccgggggggcggtttcccatgggatgtgaaaggctggccattattaagtccctgtaactcaaatgtcaaccccaccgaggcacccccccgtcccccagaatcttggctgtttacaaatcacgtgtccatcgagcacgtctgaaacccctggtagccccgacttctttttaattaaaataaggtaagcccttcaatttgtttcttcaatatttctttcatttgtagggatatttgtttttcatatcagactaataaaaagaaattagaaaccaaaaaaaaaaaaaaaaaaaa

SEQ ID NO: 6 - protein, 185 aa, Homo sapiens
  SIGNAL (1)..(22)
  mat_peptide (23)..(185)
MARGAALALLLFGLLGVLVAAPDGGFDLSDALPDNENKKPTAIPKKPSAGDDFDLGDAVVDGENDDPRPPNPPKPMPNPNPNHPSSSGSFSDADLADGVSGGEGKGGSDGGGSHRKEGEEADAPGVIPGIVGAVVVAVAGAISSFIAYQKKKLCFKENAEQGEVDMESHRNANAEPAVQRTLLEK

SEQ ID NO: 7 - DNA, 1207 bp, Homo sapiens
  sig_peptide (175)..(240)
ggaggccggggcggggcgggcgcagccggcgctgagcttgcagggccgctcccctcacccgcccccttcgagtccccgggcttcgccccacccggcccgtgggggagtatctgtcctgccgccttcgcccacgccctgcactccgggaccgtccctgcgcgctctgggcgcaccatggcccgcggggctgcgctggcgctgctgctcttcggcctgctgggtgttctggtcgccgccccggatggtggtttcgatttatccgatgcccttcctggggatgactttgacttaggagatgctgttgttgatggagaaaatgacgacccacgaccaccgaacccacccaaaccgatgccaaatccaaaccccaaccaccctagttcctccggtagcttttcagatgctgaccttgcggatggcgtttcaggtggagaaggaaaaggaggcagtgatggtggaggcagccacaggaaagaaggggaagaggccgacgccccaggcgtgatccccgggattgtgggggctgtcgtggtcgccgtggctggagccatctctagcttcattgcttaccagaaaaagaagctatgcttcaaagaaaatgcagaacaaggggaggtggacatggagagccaccggaatgccaacgcagagccagctgttcagcgtactcttttagagaaatagaagattgtcggcagaaacagcccaggcgttggcagcagggttagaacagctgcctgaggctcctccctgaaggacacctgcctgagagcagagatggaggccttctgttcacggcggattctttgttttaatcttgcgatgtgctttgcttgttgctgggcggatgatgtttactaacgatgaattttacatccaaagggggataggcacttggacccccattctccaaggcccgggggggcggtttcccatgggatgtgaaaggctggccattattaagtccctgtaactcaaatgtcaaccccaccgaggcacccccccgtcccccagaatcttggctgtttacaaatcacgtgtccatcgagcacgtctgaaacccctggtagccccgacttctttttaattaaaataaggtaagcccttcaatttgtttcttcaatatttctttcatttgtagggatatttgtttttcatatcagactaataaaaagaaattagaaaccaaaaaaaaaaaaaaaaaaaa

SEQ ID NO: 8 - protein, 169 aa, Homo sapiens
  SIGNAL (1)..(22)
  mat_peptide (23)..(169)
MARGAALALLLFGLLGVLVAAPDGGFDLSDALPGDDFDLGDAVVDGENDDPRPPNPPKPMPNPNPNHPSSSGSFSDADLADGVSGGEGKGGSDGGGSHRKEGEEADAPGVIPGIVGAVVVAVAGAISSFIAYQKKKLCFKENAEQGEVDMESHRNANAEPAVQRTLLEK

(19) United States
(12) Patent Application Publication
(10) Pub. No.: US 2012/0121588 A1
(43) Pub. Date: May 17, 2012

(54) MODULATION OF PILR RECEPTORS TO TREAT SEPSIS

(75) Inventors: Antara Banerjee, Fremont, CA (US); Paul G. Heyworth, San Francisco, CA (US)

(73) Assignee: SCHERING CORPORATION, Kenilworth, NJ (US)

(21) Appl. No.: 13/321,406

(22) PCT Filed: May 14, 2010
(86) PCT No.: PCT/US2010/034877

§ 371 Date: Feb. 1, 2012

Related U.S. Application Data
(60) Provisional application No. 61/179,981, filed on May 20, 2009.

(51) Int. Cl. A61K 39/395; A61K 31/7088; A61P 31/00; A61K 38/02

(52) U.S. Cl. 424/133.1; 424/130.1; 424/178.1; 514/1.4; 424/141.1; 514/44 A

(57) ABSTRACT

The present invention provides methods of using agonists and antagonists of PILRα and PILRβ, respectively, to treat immune mediated sepsis. Also provided are methods of prophylactically treating with agonists and antagonists of PILRα and PILRβ respectively, to prevent the development of sepsis.

### FIELD OF THE INVENTION

The present invention provides methods of modulating PILR receptors to control systemic inflammatory disorders, in particular, sepsis.

### BACKGROUND OF THE INVENTION

Sepsis is described as a potentially lethal clinical condition that develops as a result of dysregulated host response to bacterial infection. Despite significant advances in the understanding of its pathophysiology and identification novel therapeutic targets, sepsis still remains a leading cause of death in the United States (see, e.g., Martin, et al. (2003) N. Engl. J. Med. 348:1546-1554) among critically ill patients. It is now known that during an infection Toll-like receptors (TLRs) play a pivotal role in pathogen recognition and initiating an immune response which is primarily orchestrated as a consequence of interaction between the conserved bacterial component and the TLR (see, e.g., Bowie and O'Neill (2006) J. Leukoc. Biol. 67:508-514). These microbial components are known as a pathogen-associated molecular patterns (PAMPS) (see, e.g. Kopp and Medzhitov (2003) Curr. Opin. Immunol. 15:396-401; and Albiger, et al. (2007) J. Intern. Med. 261:511-528). An exaggerated response to these PAMPS resulting in severe infection, initiates the systemic inflammatory response syndrome (SIRS) through an intense inflammatory reaction characterized by the release of an arsenal of proinflammatory cytokines that cause destruction ultimately leading to multiple organ failure and death (see, e.g., Davies and Hagan (1997) Br. J. Surg. 84:920-935).

A joint consensus conference was held in 1991 by the Society of Critical Care Medicine and the American College of Chest Physicians. The disease concept of systemic inflammatory response syndrome (SIRS) was advocated at this conference. Namely, a pathological state having any two or more clinical symptoms of the four diagnostic parameters indicated below is diagnosed as the response of the body to trauma, burns, severe pancreatitis, infection or other forms of invasion:

(1) High body temperature of at least 38° C. or low body temperature below 36° C.; (2) Heart rate of at least 90 beats/minute;
(3) Respiration rate of at least 20 breaths/minute or PaCO2 (arterial blood carbon dioxide partial pressure) of less than 32 torr; and/or
(4) WBC count of at least 12,000/μl or less than 4,000/μl, or immature WBC count of at least 10% (see, e.g., et al., (1992) Chest 101:1644-1655).

In addition, septic shock is a disease accompanied by perfusion abnormalities such as low blood pressure even though an adequate amount of circulating body fluids is maintained. As sepsis progresses, there is onset of septic shock within several hours, presenting with decreased systemic peripheral vascular resistance, decreased myocardial contractile force, peripheral circulatory insufficiency, decreased blood pressure and so forth.

The clinical syndrome of sepsis or SIRS in mammals has been primarily attributed to lipopolysaccharide (LPS) or endotoxin, the major constituent of the outer membrane of Gram negative bacteria (see, e.g., Bosshart and Heinzelmann (2007) ann. N.Y. Acad. Sci. 1096:1-17). The binding of LPS to its receptor, TLR4 (see, e.g., Chow, et al. (1999) J. Biol. Chem. 274:10689-10692; and Hajjar, et al. (2002) Nat. Immunol. 3:354-359), triggers an inflammatory reaction, characterized by the release of proinflammatory mediators to eradicate the invading pathogen. Notably, TNFα is considered a pivotal mediator of septic shock along with other proinflammatory cytokines such IL-1β, IFN-γ, IL-6 and HMGB. In addition, besides these cytokines, eicosanoids such as leukotriene B4, thromboxane B2 and prostaglandins have been reported to be higher than normal, while the complement system has also been reported to be activated (Takakuwa et al., (1994) Res. Commun. Chem. Pathol. Pharmacol. 84:291-300). An excessive and uncontrolled release of these mediators leads to septic shock.

Since the nature and duration of the septic response relies on the cross talk of several cytokines as well as many receptors and adaptor molecules, several attempts towards drug design and targeted treatment of sepsis, have largely remained unsuccessful. Although much is known about the TLRs and their ability to respond to the invading pathogen, a better understanding of the biology and the role of other myeloid receptors during this acute phase of endotoxemia warrants further investigation to facilitate generation of more effective therapeutics to efficaciously manage sepsis.

Myeloid cells such as neutrophils and macrophages, which form the first line of host defense during an innate immune response, play a very important role in the progression of inflammation during sepsis. These cells express various activating and inhibitory immune regulatory receptors (see, e.g., Colonna (2003) Nat. Rev. Immunol. 3:445-453) and along with the TLRs contribute significantly towards regulating and sustaining a balance between the protective and destructive components of inflammation. The paired-immunoglobulin type 2-like receptor (PILR) family comprises both inhibitory PILRα (aka inhibitory FDF03) and activating PILRβ (aka activating FDF03) isoforms, and is well conserved among most mammals (see, e.g., Fournier, et al. (2000) J. Immunol. 165:1197-1209; and Shiratori, et al. (2004) J. Exp. Med. 525-533). Both receptors belong to the v-type immunoglobulin superfamily and are mapped to chromosome 7q22 in human. Both isoforms are expressed on neutrophils, monocytes, macrophages, and dendritic cells (see, e.g., Fournier, et al. supra).

Additionally, PILRβ is also present on NK cells and a small population of T cells in both mouse and human (see, e.g., Fournier, et al. supra; and Shiratori et al. supra). PILRα possesses two ITIM motifs in its cytoplasmic domain and delivers inhibitory signals through recruitment of SHP-1 via its amino-terminal SH2 domain (see, e.g., Mousseau et al. (2000) J. Biol. Cheml 275:4467-4474). Conversely, PILRβ, which does not contain an ITIM motif, associates with the adaptor molecule, DAP12, and transduces an activating signal through the DAP12 immunoregulatory tyrosine-based activation motif (ITAM; see, e.g., Shiratori, et al. supra). Initial studies reported CD99 to be a potential ligand for both receptors in mouse (see, e.g., Shiratori, et al. supra). However, more recently it was observed that only the O-glycan sugar chain on CD99 and not the whole CD99 molecule itself is involved in receptor recognition (see, e.g., Wang, et al. (2008) J. Immunol. 180:1686-1693). Also, recent studies have demonstrated glycoprotein-B of the herpes simplex virus-1 to be a ligand for PILRα (see, e.g., Satoh, et al. (2008) Cell 132:935-944), signifying an alternative route for viral entry into the infected cells. Most of the current knowledge regarding the PILRs comes from experiments done in NK cells and dendritic cells and attempts to unveil the ligand for these two receptors. However, their role and mechanism of activation or inhibition during an inflammatory response is not clearly understood.

Triggering receptor expressed on myeloid cells 1 (TREM-1), has been recently identified as an activating receptor of the immunoglobulin superfamily that associates with a transmembrane signaling adaptor molecule, DAP12 (see, e.g., Lanier and Bakker (2000) Immunol. Today 21:611-614; and Taylor, et al. (2005) Annu. Rev. Immunol. 23:901-944) and upon antibody ligation is capable of eliciting inflammatory responses on neutrophils and monocytes (see, e.g., Bouchon, et al. (2000) J. Immunol. 164:4991-4995). Further studies also revealed that TREM-1 plays a critical role in acute inflammatory responses to bacteria and is a crucial mediator of septic shock (see, e.g., Bouchon et al. (2001) Nature 410:1103-1107), and blockade of TREM-1 results in dampening of inflammation accompanied by increased survival and protection of the mice from septic shock. A recent study by Dower et al. has shown that the proinflammatory effect of TREM-1 activation during LPS-mediated endotoxemia is a cumulative effect of the overlap and cross-talk between the ITAM- and TLR-mediated signaling pathways (see, e.g. Dower et al. (2008) J. Immunol. 180:3520-3534). Consistent with the above findings, Turnbull et al. also demonstrated that the adaptor molecule DAP12 is also an amplifier of inflammation during acute endotoxemia (see, e.g., Turnbull et al. (2005) J. Exp. Med. 202:363-369). Furthermore, DAP12 transgenic mice exhibited increased systemic inflammation and mortality during endotoxemia (Lucas, et al. Eur J. Immunol. (2002) 32:2653-2663). In contrast, previous studies have also reported an increased response to low concentrations of microbial products in macrophages derived from DAP12−/− mice, suggesting a possible inhibitory role for DAP12 in regulating inflammation (Hamermann et al. (2005) Nat. Immunol. 6:579-586).

As has been described above, there are multiple types of factors involved in the pathogenesis of sepsis, and the disease state of sepsis is assumed to be determined through a complex relationship of these factors. Thus, a need exists for additional therapies to regulate the septic response.

### SUMMARY OF THE INVENTION

The present invention is based, in part, upon the discovery that modulating PILRα or PILRβ can affect the development or progression of sepsis.

The present invention encompasses a method of modulating sepsis comprising administering to a subject in need of such treatment, an effective amount of an antagonist of PILRβ. In one embodiment, the antagonist of PILRβ is an antibody, antibody fragment, or antibody conjugate. The antibody can be a polyclonal antibody, a monoclonal antibody, a recombinant antibody, a humanized antibody or fragment thereof, a fully human antibody or fragment thereof. The antagonist can also be a soluble PILRβ polypeptide, or a soluble PILRβ polypeptide fused to a heterologous protein. For example, a soluble PILRβ polypeptide or fusion polypeptide may comprise residues 20-191 of SEQ ID NO: 4. The antagonist of PILRβ reduces at least one symptom of sepsis. In a further embodiment, the antagonist of PILRβ is administered with at least one antibiotic having bateriocidal or bacteriostatic activity.

The present invention encompasses a method of modulating sepsis comprising administering to a subject in need of such treatment, an effective amount of an agonist of PILRα. In one embodiment, the antagonist of PILRα is an antibody, antibody fragment, or antibody conjugate. The antibody can be a polyclonal antibody, a monoclonal antibody, a recombinant antibody, a humanized antibody or fragment thereof, a fully human antibody or fragment thereof. The agonist of PILRα reduces at least one symptom of sepsis. In one embodiment, the agonist of PILRα is administered with at least one antibiotic having bateriocidal or bacteriostatic activity.

The present invention provides a method of prophylactically treating a subject to prevent sepsis comprising administering to the subject in need of such treatment, an effective amount of an antagonist of PILRβ. In one embodiment, the antagonist of PILRβ is an antibody, antibody fragment, or antibody conjugate, including a polyclonal antibody, a monoclonal antibody, a recombinant antibody, a humanized antibody or fragment thereof, a fully human antibody or fragment thereof. The antagonist can also be a soluble PILRβ polypeptide, or a soluble PILRβ polypeptide fused to a heterologous protein. For example, a soluble PILRβ polypeptide or fusion polypeptide may comprise residues 20-191 of SEQ ID NO: 4. The antagonist of PILRβ prevents at least one symptom of sepsis. In one embodiment, the antagonist of PILRβ is administered with at least one antibiotic having bateriocidal or bacteriostatic activity.

The present invention provides a method of prophylactically treating a subject against sepsis comprising administering to the subject in need of such treatment, an effective amount of an agonist of PILRα. In one embodiment, the agonist of PILRα is antibody, antibody fragment or antibody conjugate, including a polyclonal antibody, a monoclonal antibody, a recombinant antibody, a humanized antibody or fragment thereof, a fully human antibody or fragment thereof. The agonist of PILRα prevents at least one symptom of sepsis. In a further embodiment, the agonist of PILRα is administered with at least one antibiotic having bateriocidal or bacteriostatic activity.

In other embodiments the antagonist of PILRβ comprises a polynucleotide. In various embodiments the polynucleotide is an antisense nucleic acid (e.g. antisense RNA) or an interfering nucleic acid, such as a small interfering RNA (siRNA). In one embodiment the polynucleotide antagonist of PILRβ is delivered in gene therapy vector, such as an adenovirus, lentivirus, retrovirus or adenoassociated virus vector. In another embodiment the polynucleotide antagonist of PILRβ is delivered as a therapeutic agent.

### DETAILED DESCRIPTION

As used herein, including the appended claims, the singular forms of words such as “a,” “an,” and “the,” include their corresponding plural references unless the context clearly dictates otherwise.

All references cited herein are incorporated by reference to the same extent as if each individual publication, patent application, or patent, was specifically and individually indicated to be incorporated by reference.

### I. DEFINITIONS

“Activity” of a molecule may describe or refer to the binding of the molecule to a ligand or to a receptor, to catalytic activity, to the ability to stimulate gene expression, to antigenic activity, to the modulation of activities of other molecules, and the like. “Activity” of a molecule may also refer to activity in modulating or maintaining cell-to-cell interactions, e.g., adhesion, or activity in maintaining a structure of a cell, e.g., cell membranes or cytoskeleton. “Activity” may also mean specific activity, e.g., [catalytic activity]/[mg protein], or [immunological activity]/[mg protein], or the like.

As used herein, the phrase “pathogenic agent” means an agent that causes a disease state or affliction in an animal. Included within this definition, for example, are bacteria, protozoans, fungi, viruses and metazoan parasites which either produce a disease state or render an animal infected with such an organism susceptible to a disease state (e.g., a secondary infection). Further included are species and strains of the genus Staphylococcus which produce disease states in animals.

As used herein, the term “organism” means any living biological system, including viruses, regardless of whether it is a pathogenic agent.

As used herein, “bacteremia” means the presence of viable bacteria in the blood or organs of an individual (human or other animal).

Herein, “mammal” means human, bovine, goat, rabbit, mouse, rat, hamster, and guinea pig; preferred is human, rabbit, rat, hamster, or mouse and particularly preferred is human, rat, hamster, or mouse.

The term “mammals other than humans” and “non-human mammals” used herein, are synomic to each other, meaning all mammals other than humans defined above.

The terms “PILRα or PILRβ”, “Paired-immunoglobulin type 2-like receptor α or β”, “FDF03 inhibitory receptor and FDF03 activating receptor” are well known in the art. “PILR” will be used to represent “PILRα and PILRβ” unless otherwise specified. The human and mouse PILRα and PILRβ nucleotide and polypeptide sequences are disclosed in WO 1998/024906 and WO 2000/040721, respectively. The nucleic acid and amino acid sequences for human PILRα are also provided at SEQ ID NOs: 1 and 2, respectively. The nucleic acid and amino acid sequences for human PILRβ are provided at SEQ ID NOs: 3 and 4, respectively. Unless otherwise indicated or clear from the context, antibodies to PILRα and PILRβ, such as antibodies used in the experiments reported herein, are agonist antibodies, rather than antagonist antibodies.

“Antagonists of PILRβ activity” as used herein, applies to antibodies, antibody fragments, soluble domains of PILRβ, PILRβ fusion proteins, etc., that can inhibit the biological results of PILRβ activation. Fusion proteins are usually the soluble domain polypeptide of PILRβ associated with a heterologous protein or synthetic molecule, e.g., the Ig domain of an immunoglobulin.

“Administration” and “treatment,” as it applies to an animal, human, experimental subject, cell, tissue, organ, or biological fluid, refers to contact of an exogenous pharmaceutical, therapeutic, diagnostic agent, or composition to the animal, human, subject, cell, tissue, organ, or biological fluid. “Administration” and “treatment” can refer, e.g., to therapeutic, pharmacokinetic, diagnostic, research, and experimental methods. Treatment of a cell encompasses contact of a reagent to the cell, as well as contact of a reagent to a fluid, where the fluid is in contact with the cell. “Administration” and “treatment” also means in vitro and ex vivo treatments, e.g., of a cell, by a reagent, diagnostic, binding composition, or by another cell. “Treatment,” as it applies to a human, veterinary, or research subject, refers to therapeutic treatment, prophylactic or preventative measures, to research and diagnostic applications. “Treatment” as it applies to a human, veterinary, or research subject, or cell, tissue, or organ, encompasses contact of an agent with animal subject, a cell, tissue, physiological compartment, or physiological fluid. “Treatment of a cell” also encompasses situations where the agent contacts PILR, e.g., in the fluid phase or colloidal phase, but also situations where the agonist or antagonist does not contact the cell or the receptor.

As used herein, the term “antibody,” when used in a general sense, refers to any form of antibody that exhibits the desired biological activity. Thus, it is used in the broadest sense and specifically covers monoclonal antibodies (including full length monoclonal antibodies), polyclonal antibodies, multispecific antibodies (e.g., bispecific antibodies), chimeric antibodies, humanized antibodies, fully human antibodies, etc. so long as they exhibit the desired biological activity.

As used herein, the terms “PILR binding fragment,” “binding fragment thereof” or “antigen binding fragment thereof” encompass a fragment or a derivative of an antibody that still substantially retains its biological activity of either stimulating PILRα activity or inhibiting PILRβ activity, such inhibition being referred to herein as “PILR modulating activity.” The term “antibody fragment” or PILR binding fragment refers to a portion of a full length antibody, generally the antigen binding or variable region thereof. Examples of antibody fragments include Fab, Fab′, F(ab′)₂, and Fv fragments; diabodies; linear antibodies; single-chain antibody molecules, e.g., sc-Fv; and multispecific antibodies formed from antibody fragments. Typically, a binding fragment or derivative retains at least 10% of its MDL-1 inhibitory activity. Preferably, a binding fragment or derivative retains at least 25%, 50%, 60%, 70%, 80%, 90%, 95%, 99% or 100% (or more) of its PILR activity, although any binding fragment with sufficient affinity to exert the desired biological effect will be useful. It is also intended that a PILR binding fragment can include variants having conservative amino acid substitutions that do not substantially alter its biologic activity.

The term “monoclonal antibody”, as used herein, refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic epitope. In contrast, conventional (polyclonal) antibody preparations typically include a multitude of antibodies directed against (or specific for) different epitopes. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al. (1975) Nature 256: 495, or may be made by recombinant DNA methods (see, e.g., U.S. Pat. No. 4,816,567). The “monoclonal antibodies” may also be isolated from phage antibody libraries using the techniques described in Clackson et al. (1991) Nature 352: 624-628 and Marks et al. (1991) J. Mol. Biol. 222: 581-597, for example.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity. U.S. Pat. No. 4,816,567; Morrison et al. (1984) Proc. Natl. Acad. Sci. USA 81: 6851-6855.

A “domain antibody” is an immunologically functional immunoglobulin fragment containing only the variable region of a heavy chain or the variable region of a light chain. In some instances, two or more V_{H} regions are covalently joined with a peptide linker to create a bivalent domain antibody. The two V_{H} regions of a bivalent domain antibody may target the same or different antigens.

A “bivalent antibody” comprises two antigen binding sites. In some instances, the two binding sites have the same antigen specificities. However, bivalent antibodies may be bispecific (see below).

As used herein, the term “single-chain Fv” or “scFv” antibody refers to antibody fragments comprising the V_{H} and V_{L} domains of antibody, wherein these domains are present in a single polypeptide chain. Generally, the Fv polypeptide further comprises a polypeptide linker between the V_{H} and V_{L} domains which enables the sFv to form the desired structure for antigen binding. For a review of sFv, see Pluckthun (1994) THE PHARMACOLOGY OF MONOCLONAL ANTIBODIES, vol. 113, Rosenburg and Moore eds. Springer-Verlag, New York, pp. 269-315.

The monoclonal antibodies herein also include camelized single domain antibodies. See, e.g., Muyldermans et al. (2001) Trends Biochem. Sci. 26:230; Reichmann et al. (1999) J. Immunol. Methods 231:25; WO 94/04678; WO 94/25591; U.S. Pat. No. 6,005,079). In one embodiment, the present invention provides single domain antibodies comprising two V_{H} domains with modifications such that single domain antibodies are formed.

As used herein, the term “diabodies” refers to small antibody fragments with two antigen-binding sites, which fragments comprise a heavy chain variable domain (V_{H}) connected to a light chain variable domain (V_{L}) in the same polypeptide chain (V_{H}-V_{L} or V_{L}-V_{H}). By using a linker that is too short to allow pairing between the two domains on the same chain, the domains are forced to pair with the complementary domains of another chain and create two antigen-binding sites. Diabodies are described more fully in, e.g., EP 404,097; WO 93/11161; and Holliger et al. (1993) Proc. Natl. Acad. Sci. USA 90: 6444-6448. For a review of engineered antibody variants generally see Holliger and Hudson (2005) Nat. Biotechnol. 23:1126-1136.

As used herein, the term “humanized antibody” refers to forms of antibodies that contain sequences from non-human (e.g., murine) antibodies as well as human antibodies. Such antibodies contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. The prefix “hum”, “hu” or “h” is added to antibody clone designations when necessary to distinguish humanized antibodies from parental rodent antibodies. The humanized forms of rodent antibodies will generally comprise the same CDR sequences of the parental rodent antibodies, although certain amino acid substitutions may be included to increase affinity, increase stability of the humanized antibody, or for other reasons.

The antibodies of the present invention also include antibodies with modified (or blocked) Fc regions to provide altered effector functions. See, e.g., U.S. Pat. No. 5,624,821; WO2003/086310; WO2005/120571; WO2006/0057702; Presta (2006) Adv. Drug Delivery Rev. 58:640-656. Such modification can be used to enhance or suppress various reactions of the immune system, with possible beneficial effects in diagnosis and therapy. Alterations of the Fc region include amino acid changes (substitutions, deletions and insertions), glycosylation or deglycosylation, and adding multiple Fc. Changes to the Fc can also alter the half-life of antibodies in therapeutic antibodies, and a longer half-life would result in less frequent dosing, with the concomitant increased convenience and decreased use of material. See Presta (2005) J. Allergy Clin. Immunol. 116:731 at 734-35.

The antibodies of the present invention also include antibodies with intact Fc regions that provide full effector functions, e.g. antibodies of isotype IgG1, which induce complement-dependent cytotoxicity (CDC) or antibody dependent cellular cytotoxicity (ADCC) in the a targeted cell.

The antibodies of the present invention also include antibodies conjugated to cytotoxic payloads, such as cytotoxic agents or radionuclides. Such antibody conjugates may be used in immunotherapy to selectively target and kill cells expressing MDL-1 and/or DAP12 on their surface. Exemplary cytotoxic agents include ricin, vinca alkaloid, methotrexate, Psuedomonas exotoxin, saporin, diphtheria toxin, cisplatin, doxorubicin, abrin toxin, gelonin and pokeweed antiviral protein. Exemplary radionuclides for use in immunotherapy with the antibodies of the present invention include ¹²⁵I, ¹³¹I, ⁹⁰Y, ⁶⁷Cu, ²¹¹At, ¹⁷⁷Lu, ¹⁴³Pr and ²¹³Bi. See, e.g., U.S. Patent Application Publication No. 2006/0014225.

The term “fully human antibody” refers to an antibody that comprises human immunoglobulin protein sequences only. A fully human antibody may contain murine carbohydrate chains if produced in a mouse, in a mouse cell, or in a hybridoma derived from a mouse cell. Similarly, “mouse antibody” or “rat antibody” refer to an antibody that comprises only mouse or rat immunoglobulin sequences, respectively. A fully human antibody may be generated in a human being, in a transgenic animal having human immunoglobulin germline sequences, by phage display or other molecular biological methods.

As used herein, the term “hypervariable region” refers to the amino acid residues of an antibody that are responsible for antigen-binding. The hypervariable region comprises amino acid residues from a “complementarity determining region” or “CDR” (e.g. residues 24-34 (CDRL1), 50-56 (CDRL2) and 89-97 (CDRL3) in the light chain variable domain and residues 31-35 (CDRH1), 50-65 (CDRH2) and 95-102 (CDRH3) in the heavy chain variable domain (Kabat et al. (1991) Sequences of Proteins of Immunological Interest, 5th Ed. Public Health Service, National Institutes of Health, Bethesda, Md.) and/or those residues from a “hypervariable loop” (i.e. residues 26-32 (L1), 50-52 (L2) and 91-96 (L3) in the light chain variable domain and 26-32 (H1), 53-55 (H2) and 96-101 (H3) in the heavy chain variable domain (Chothia and Lesk (1987) J. Mol. Biol. 196: 901-917). As used herein, the term “framework” or “FR” residues refers to those variable domain residues other than the hypervariable region residues defined herein as CDR residues. The residue numbering above relates to the Kabat numbering system and does not necessarily correspond in detail to the sequence numbering in the accompanying Sequence Listing.

“Binding compound” refers to a molecule, small molecule, macromolecule, polypeptide, antibody or fragment or analogue thereof, or soluble receptor, capable of binding to a target. “Binding compound” also may refer to a complex of molecules, e.g., a non-covalent complex, to an ionized molecule, and to a covalently or non-covalently modified molecule, e.g., modified by phosphorylation, acylation, cross-linking, cyclization, or limited cleavage, that is capable of binding to a target. When used with reference to antibodies, the term “binding compound” refers to both antibodies and antigen binding fragments thereof. “Binding” refers to an association of the binding composition with a target where the association results in reduction in the normal Brownian motion of the binding composition, in cases where the binding composition can be dissolved or suspended in solution. “Binding composition” refers to a molecule, e.g. a binding compound, in combination with a stabilizer, excipient, salt, buffer, solvent, or additive, capable of binding to a target.

“Conservatively modified variants” or “conservative substitution” refers to substitutions of amino acids are known to those of skill in this art and may often be made even in essential regions of the polypeptide without altering the biological activity of the resulting molecule. Such exemplary substitutions are preferably made in accordance with those set forth in Table 1 as follows:

TABLE 1
Exemplary Conservative Amino Acid Substitutions

| Original | Conservative |
|---|---|
| residue | substitution |
| Ala (A) | Gly; Ser |
| Arg (R) | Lys, His |
| Asn (N) | Gln; His |
| Asp (D) | Glu; Asn |
| Cys (C) | Ser; Ala |
| Gln (Q) | Asn |
| Glu (E) | Asp; Gln |
| Gly (G) | Ala |
| His (H) | Asn; Gln |
| Ile (I) | Leu; Val |
| Leu (L) | Ile; Val |
| Lys (K) | Arg; His |
| Met (M) | Leu; Ile; Tyr |
| Phe (F) | Tyr; Met; Leu |
| Pro (P) | Ala |
| Ser (S) | Thr |
| Thr (T) | Ser |
| Trp (W) | Tyr; Phe |
| Tyr (Y) | Trp; Phe |
| Val (V) | Ile; Leu |

Those of skill in this art recognize that, in general, single amino acid substitutions in non-essential regions of a polypeptide may not substantially alter biological activity. See, e.g., Watson et al. (1987) Molecular Biology of the Gene, The Benjamin/Cummings Pub. Co., p. 224 (4th Edition).

The phrase “consists essentially of,” or variations such as “consist essentially of” or “consisting essentially of,” as used throughout the specification and claims, indicate the inclusion of any recited elements or group of elements, and the optional inclusion of other elements, of similar or different nature than the recited elements, that do not materially change the basic or novel properties of the specified dosage regimen, method, or composition. As a non-limiting example, a binding compound that consists essentially of a recited amino acid sequence may also include one or more amino acids, including substitutions of one or more amino acid residues, that do not materially affect the properties of the binding compound.

“Effective amount” encompasses an amount sufficient to ameliorate or prevent a symptom or sign of the medical condition. Effective amount also means an amount sufficient to allow or facilitate diagnosis. An effective amount for a particular patient or veterinary subject may vary depending on factors such as the condition being treated, the overall health of the patient, the method route and dose of administration and the severity of side affects. See, e.g., U.S. Pat. No. 5,888,530. An effective amount can be the maximal dose or dosing protocol that avoids significant side effects or toxic effects. The effect will result in an improvement of a diagnostic measure or parameter by at least 5%, usually by at least 10%, more usually at least 20%, most usually at least 30%, preferably at least 40%, more preferably at least 50%, most preferably at least 60%, ideally at least 70%, more ideally at least 80%, and most ideally at least 90%, where 100% is defined as the diagnostic parameter shown by a normal subject. See, e.g., Maynard et al. (1996) A Handbook of SOPs for Good Clinical Practice, Interpharm Press, Boca Raton, Fla.; Dent (2001) Good Laboratory and Good Clinical Practice, Urch Publ., London, UK.

“Immune condition” or “immune disorder” encompasses, e.g., pathological inflammation, an inflammatory disorder, and an autoimmune disorder or disease. “Immune condition” also refers to infections, persistent infections, and proliferative conditions, such as cancer, tumors, and angiogenesis, including infections, tumors, and cancers that resist eradication by the immune system. “Cancerous condition” includes, e.g., cancer, cancer cells, tumors, angiogenesis, and precancerous conditions such as dysplasia.

“Infection” as used herein is an invasion and multiplication of microorganisms in tissues of a subject's body. The infection or “infectious disease” may be clinically inapparent or result in local cellular injury due to competitive metabolism, toxins, intracellular replication, or antigen-antibody response. The infection may remain localized, subclinical and temporary if the body's defensive mechanisms are effective. A local invention may persist and spread by extension to become an acute, subacute, or chronic clinical infection or disease state. A local infection may also become systemic when the microorganisms gain access to the lymphatic or vascular system. Infectious diseases include bacterial, viral, parasitic, opportunistic, or fungal infections.

As used herein “antibiotic” refers to an aminoglycoside such as gentamycin or a beta-lactam such as penicillin, cephalosporin and the like. Also included are known anti-fungals and anti-virals. Antibiotics can be used with the MDL-1 antibodies of the present invention to provide additional efficacy to clear the infection and/or prevent the development of sepsis.

As used herein, the term “isolated nucleic acid molecule” refers to a nucleic acid molecule that is identified and separated from at least one contaminant nucleic acid molecule with which it is ordinarily associated in the natural source of the antibody nucleic acid. An isolated nucleic acid molecule is other than in the form or setting in which it is found in nature. Isolated nucleic acid molecules therefore are distinguished from the nucleic acid molecule as it exists in natural cells. However, an isolated nucleic acid molecule includes a nucleic acid molecule contained in cells that ordinarily express the antibody where, for example, the nucleic acid molecule is in a chromosomal location different from that of natural cells.

The expression “control sequences” refers to DNA sequences involved in the expression of an operably linked coding sequence in a particular host organism. The control sequences that are suitable for prokaryotes, for example, include a promoter, optionally an operator sequence, and a ribosome binding site. Eukaryotic cells are known to use promoters, polyadenylation signals, and enhancers.

A nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading frame. However, enhancers do not have to be contiguous. Linking is accomplished by ligation at convenient restriction sites. If such sites do not exist, the synthetic oligonucleotide adaptors or linkers are used in accordance with conventional practice.

As used herein, the expressions “cell,” “cell line,” and “cell culture” are used interchangeably and all such designations include progeny. Thus, the words “transformants” and “transformed cells” include the primary subject cell and cultures derived therefrom without regard for the number of transfers. It is also understood that all progeny may not be precisely identical in DNA content, due to deliberate or inadvertent mutations. Mutant progeny that have the same function or biological activity as screened for in the originally transformed cell are included. Where distinct designations are intended, it will be clear from the context.

As used herein, “polymerase chain reaction” or “PCR” refers to a procedure or technique in which minute amounts of a specific piece of nucleic acid, RNA and/or DNA, are amplified as described in, e.g., U.S. Pat. No. 4,683,195. Generally, sequence information from the ends of the region of interest or beyond needs to be available, such that oligonucleotide primers can be designed; these primers will be identical or similar in sequence to opposite strands of the template to be amplified. The 5′ terminal nucleotides of the two primers can coincide with the ends of the amplified material. PCR can be used to amplify specific RNA sequences, specific DNA sequences from total genomic DNA, and cDNA transcribed from total cellular RNA, bacteriophage or plasmid sequences, etc. See generally Mullis et al. (1987) Cold Spring Harbor Symp. Quant. Biol. 51:263; Erlich, ed., (1989) PCR TECHNOLOGY (Stockton Press, N.Y.) As used herein, PCR is considered to be one, but not the only, example of a nucleic acid polymerase reaction method for amplifying a nucleic acid test sample comprising the use of a known nucleic acid as a primer and a nucleic acid polymerase to amplify or generate a specific piece of nucleic acid.

As used herein, the term “germline sequence” refers to a sequence of unrearranged immunoglobulin DNA sequences, including rodent (e.g. mouse) and human germline sequences. Any suitable source of unrearranged immunoglobulin DNA may be used. Human germline sequences may be obtained, for example, from JOINSOLVER® germline databases on the website for the National Institute of Arthritis and Musculoskeletal and Skin Diseases of the United States National Institutes of Health. Mouse germline sequences may be obtained, for example, as described in Giudicelli et al. (2005) Nucleic Acids Res. 33:D256-D261.

To examine the extent of modulation of PILR activity, for example, samples or assays comprising a given, e.g., protein, gene, cell, or organism, are treated with a potential activating or inhibiting agent and are compared to control samples without the agent. Control samples, i.e., not treated with agent, are assigned a relative activity value of 100%. Inhibition is achieved when the activity value relative to the control is about 90% or less, typically 85% or less, more typically 80% or less, most typically 75% or less, generally 70% or less, more generally 65% or less, most generally 60% or less, typically 55% or less, usually 50% or less, more usually 45% or less, most usually 40% or less, preferably 35% or less, more preferably 30% or less, still more preferably 25% or less, and most preferably less than 20%. Activation is achieved when the activity value relative to the control is about 110%, generally at least 120%, more generally at least 140%, more generally at least 160%, often at least 180%, more often at least 2-fold, most often at least 2.5-fold, usually at least 5-fold, more usually at least 10-fold, preferably at least 20-fold, more preferably at least 40-fold, and most preferably over 40-fold higher.

Endpoints in activation or inhibition can be monitored as follows. Activation, inhibition, and response to treatment, e.g., of a cell, physiological fluid, tissue, organ, and animal or human subject, can be monitored by an endpoint. The endpoint may comprise a predetermined quantity or percentage of, e.g., an indicia of inflammation, oncogenicity, or cell degranulation or secretion, such as the release of a cytokine, toxic oxygen, or a protease. The endpoint may comprise, e.g., a predetermined quantity of ion flux or transport; cell migration; cell adhesion; cell proliferation; potential for metastasis; cell differentiation; and change in phenotype, e.g., change in expression of gene relating to inflammation, apoptosis, transformation, cell cycle, or metastasis (see, e.g., Knight (2000) Ann. Clin. Lab. Sci. 30:145-158; Hood and Cheresh (2002) Nature Rev. Cancer 2:91-100; Timme et al. (2003) Curr. Drug Targets 4:251-261; Robbins and Itzkowitz (2002) Med. Clin. North Am. 86:1467-1495; Grady and Markowitz (2002) Annu. Rev. Genomics Hum. Genet. 3:101-128; Bauer, et al. (2001) Glia 36:235-243; Stanimirovic and Satoh (2000) Brain Pathol. 10:113-126).

An endpoint of inhibition is generally 75% of the control or less, preferably 50% of the control or less, more preferably 25% of the control or less, and most preferably 10% of the control or less. Generally, an endpoint of activation is at least 150% the control, preferably at least two times the control, more preferably at least four times the control, and most preferably at least 10 times the control.

“Small molecule” is defined as a molecule with a molecular weight that is less than 10 kDa, typically less than 2 kDa, and preferably less than 1 kDa. Small molecules include, but are not limited to, inorganic molecules, organic molecules, organic molecules containing an inorganic component, molecules comprising a radioactive atom, synthetic molecules, peptide mimetics, and antibody mimetics. As a therapeutic, a small molecule may be more permeable to cells, less susceptible to degradation, and less apt to elicit an immune response than large molecules. Small molecules, such as peptide mimetics of antibodies and cytokines, as well as small molecule toxins are described. See, e.g., Casset et al. (2003) Biochem. Biophys. Res. Commun. 307:198-205; Muyldermans (2001) J. Biotechnol. 74:277-302; Li (2000) Nat. Biotechnol. 18:1251-1256; Apostolopoulos et al. (2002) Curr. Med. Chem. 9:411-420; Monfardini et al. (2002) Curr. Pharm. Des. 8:2185-2199; Domingues et al. (1999) Nat. Struct. Biol. 6:652-656; Sato and Sone (2003) Biochem. J. 371:603-608; U.S. Pat. No. 6,326,482.

“Specifically” or “selectively” binds, when referring to a ligand/receptor, antibody/antigen, or other binding pair, indicates a binding reaction that is determinative of the presence of the protein in a heterogeneous population of proteins and other biologics. Thus, under designated conditions, a specified ligand binds to a particular receptor and does not bind in a significant amount to other proteins present in the sample. As used herein, an antibody is said to bind specifically to a polypeptide comprising a given sequence (in this case PILR) if it binds to polypeptides comprising the sequence of PILR but does not bind to proteins lacking the sequence of PILR. For example, an antibody that specifically binds to a polypeptide comprising PILR may bind to a FLAG®-tagged form of PILR but will not bind to other FLAG®-tagged proteins.

The antibody, or binding composition derived from the antigen-binding site of an antibody, of the contemplated method binds to its antigen with an affinity that is at least two fold greater, preferably at least ten times greater, more preferably at least 20-times greater, and most preferably at least 100-times greater than the affinity with unrelated antigens. In a preferred embodiment the antibody will have an affinity that is greater than about 10⁹ liters/mol, as determined, e.g., by Scatchard analysis. Munsen et al. (1980) Analyt. Biochem. 107:220-239.

### II. General

The present invention provides methods of modulating host defense with agonists or antagonists of PILRα and PILRβ, in particular, treatment and/or prevention of sepsis or septic shock.

An uncontrolled systemic inflammation in response to microbial pathogens often results in septic shock and is a cause for high mortality among patients in the intensive care unit. Neutrophils and macrophages play a very critical role in the first line of host defense through a tight regulation between pattern recognition receptors and different activating and inhibitory receptors expressed on their cell surfaces. The paired Ig-like type 2 receptors, which comprise both the activating (PILRβ) and inhibitory (PILRα) isoforms have been recently identified on myeloid cells. However, their role during an acute inflammatory response remain to be defined. The data described below show that triggering of PILRβ with an agonist mAb results in high TNFα levels and increased mortality, whereas mice that received anti-PILRα mAb or the isotype control displayed lower serum TNFα levels and succumbed more slowly to LPS-mediated endotoxic shock. To assess a direct role for PILRβ signaling in vivo, the response of WT and PILRβ−/− mice to septic shock and peritonitis was measured. The results show that PILRβ−/− mice are highly resistant to LPS as well as cecal ligation and puncture induced septic shock compared to the WT animals and exhibit significantly reduced proinflammatory serum cytokine levels. However, PILRβ signaling did not affect cellular recruitment or bacterial control during the acute phase response of sepsis. Taken together the data demonstrates a critical function of PILRβ in the acute inflammatory response, and provides a new perspective on PILRβ and/or PILRα as novel therapeutic targets for septic shock.

A. Regulation of Human PILRα and PILRβ Surface Expression

Using in-house human monoclonal agonist antibodies to PILRα and PILRβ, FACS staining shows both PILRα and PILRβ are constitutively expressed on human neutrophils, macrophages and DC's and the expression of both PILRα and β are strongly upregulated after incubation of human neutrophils with LPS. The strong upregulation in expression levels of both PILRα and β suggests a possible synergy between the PILRs and TLRs and their involvement in the pathogenesis of sepsis.

B. Activation of PILRβ Augments Susceptibility to LPS-Mediated Endotoxemia.

To determine if PILRα and PILRβ contributed to in vivo responses to endotoxin, C57BL/6J mice were subjected to s.c.doses (1 mg/mouse) of isotype control, as well as anti-PILRα and anti-PILRβ antibodies 24 h prior to i.p injections of LPS. Mice treated with anti-PILRβ antibody showed increased susceptibility to LPS shock and succumbed faster with a 100% mortality rate within 45 h post LPS administration compared to the isotype treated group which displayed only 65% mortality even at 100 h post LPS insult. Conversely, survival in the anti-PILRα treated animals was not significantly different from that of the control group and a mortality rate of 80% was observed after 4 days of LPS insult.

Consistent with increased mortality, 1 h post LPS treatment, serum TNFα levels were also higher in the anti-PILRβ treated mice, compared to the anti-PILRα and isotype treated groups. Injection of the antibodies alone in normal mice did not result in an induction of the proinflammatory cytokine response measured at different time points (data not shown), suggesting that the profound burst in TNFα levels within 1 h post LPS administration was entirely a result of the endotoxic insult and possible increase in the number of circulating neutrophils and macrophages infiltrating the peritoneum. Mice were also injected with a lower dose of agonistic antibody, i.e. 600 μg/mouse, with the LPS level kept constant. In this experiment, the anti-PILRβ treated mice displayed 90% mortality 4 days after LPS administration. These mice also had significantly higher TNFα levels compared to the isotype control treated animals but the TNFα concentrations were lower compared to 17 ng/ml in mice that received 1 mg of anti-PILRβ antibody (see, Table 2).

TABLE 2
TNFα levels 24 hours post LPS treatment

| Isotype | anti-PILRβ | anti-PILRα |
|---|---|---|
| 2373.2 | 17112 | 1192 |
| 3712.6 | 25000 | 3835.7 |
| 3540 | 26000 | 17017.4 |
| 7964 | 15193 | 7252 |
| 5770 | 7474 | 3019 |
| 6578 | 11751 | 7618 |
| 4075 | 6510 | 4756 |
| 6827 | 12781 | 8369 |

These results suggest that activation of PILRβ with the agonistic antibody is dose-dependent and its association with DAP12 can lead to an overactivation and increased proinflammtory response. Even though PILRα and PILRβ are opposing receptor pairs, in this model of endotoxic shock, the proinflammatory response observed upon activation of PILRβ seems to predominate over the inhibitory signal of PILRα, suggesting a critical role for PILRβ in amplifying the inflammatory response during LPS-mediated endotoxemia.

C. Abrogation of PILRβ Renders Mice Completely Resistant to LPS-Mediated Septic Shock

Although TLR4 has been identified as an essential receptor for LPS signaling, the TLR4 receptor alone is unable to confer LPS responsiveness and requires appropriate association with receptors and proteins such as CD14, MD2 to transmit the LPS signal (see, e.g., Fujihara, et al. (2003) Pharmacol. Ther. 100:171-194; and Nagai, et al. (2002) Nat. Immunol. 3:667-672). Additionally, many genetically modified mouse models lacking TLR4-mediated pathway molecules were found to be resistant to LPS-induced endotoxic shock.

WT and PILRβ−/− mice were subjected to LPS challenge. The mice received either of two concentrations (180 ug/mouse or 250 ug/mouse) of LPS. LPS was administered i.p. and the mice were monitored for survival. PILRβ deficient mice displayed resistance to the LPS treatment at both concentrations of LPS and displayed 100% survival. On the other hand, the WT mice highly susceptible to LPS at both the low dose of 180 μg and higher dose of 250 μg. At the lower dose of 180 μg, the WT mice exhibited 80% mortality at 60 h post LPS injection and the mice started to succumb only after the first 20 h. At the higher dose of LPS however, 100% mortality of the WT mice was observed by 65 h and the mice submitted to the LPS insult within 15 h of LPS administration.

The proinflammatory cytokine response post LPS injection in WT and PILRβ-−/− mice was also determined. Consistent with the survival data, deletion of PILRβ resulted in mice having reduced levels of almost all of the serum proinflammatory mediators. Although, 1 h post LPS injection both WT and PILRβ−/− mice had appreciable levels of TNFα, IL-12p70, IL-1β, IL-1α, IFN-γ and MCP-1, the levels of TNFα, IL-12p70, IL-10 and MCP-1 were significantly higher in the WT compared to the knockout mice. Proinflammatory cytokines IL-1β and IFN-γ, which peak between 4 to 6 h after infliction of an insult, were found to be present at even higher levels at 4 h post LPS injection in both WT and PILRβ deficient mice, but still the levels were significantly higher in WT mice compared to the knockout mice. Levels of IL-6 were similar between the two groups at 1 h, but 4 h after injection a decreasing trend was observed in the knockout mice, although not significantly different. Interestingly, IL-10 levels were found to be significantly higher in the PILRβ−/− mice after 1 h post LPS injection and by 4 h the levels were reduced and found to be similar between the two groups. This suggests that the initial release and increase in the proinflammatory response is counteracted very efficiently by an equally significant anti-inflamamtory surge from increased levels of IL-10 observed in case of the PILRβ−/− mice. In the WT mice, the levels of IL-10 were not able to suppress the explosion of proinflammatory mediators, resulting in increased susceptibility to LPS-induced endotoxic shock (see Table 3).

TABLE 3
Serum Cytokine Levels post LPS treatment

| | |
|---|---|
| WT | PILRβ−/− |

| Cytokine | 1 h | 4 h | 1 h | 4 h |
|---|---|---|---|---|
| TNFα | 12016 | 272.92 | 1696 | 27.37 |
| | 2647 | 215.91 | 2132 | 25.35 |
| | 11884 | 122.25 | 1971 | 364.69 |
| | 5915 | 201.99 | 2450 | 361.41 |
| | 4581 | 400.2 | 2198 | 288.61 |
| | 6566 | 376.58 | 2707 | 314.78 |
| | | 182.12 | | 277.31 |
| | | 2.7 | | 0.76 |
| | | 3.2 | | 139.36 |
| IFNg | 39.02 | 796.54 | 31.69 | 3.2 |
| | 54.59 | 548.16 | 42.58 | 8.81 |
| | 41.99 | 1595.06 | 52.35 | 288.56 |
| | 74.53 | 1170.92 | 23.31 | 381.07 |
| | 41.99 | 73.79 | 24.64 | 142.86 |
| | | 472.9 | | 97.88 |
| | | 147.68 | | 232.34 |
| | | 3.65 | | 7.47 |
| | | 3.2 | | 810.2 |
| MCP-1 | 5070.37 | 24537.4 | 1267.16 | 5184.62 |
| | 14668.03 | 12538.63 | 10000 | 6788.21 |
| | 17455.45 | 3145.01 | 3.2 | 10773.19 |
| | 5082.83 | 21806.1 | 3.2 | 24354.87 |
| | 7495.34 | 16941.01 | 8083.02 | 30106.19 |
| | 11172.4 | 23969.1 | 11617.4 | 20719.92 |
| | 12774.5 | 18917.7 | 5374.19 | 20866.79 |
| | 10894.3 | | 11222.4 | 61.03 |
| | 12391.4 | | 11067.9 | 276.08 |
| | 12490.5 | | 9842.42 | |
| IL-1β | 6.62 | 309.8 | 5.75 | 15.41 |
| | 9.96 | 166.78 | 5.3 | 3.2 |
| | 7.9 | 81.77 | 16.93 | 286.26 |
| | 5.75 | 226.7 | 2.97 | 40.04 |
| | 4.85 | 219.89 | 2.47 | 114.7 |
| | | 340.68 | | 175.6 |
| | | 86.51 | | 121.69 |
| | | 13.19 | | 3.2 |
| IL-6 | 26963.46 | 13418.79 | 24958.76 | 457.16 |
| | 25418.88 | 13314.51 | 26020.28 | 206.58 |
| | 27592.2 | 964.19 | 28185.44 | 13343.96 |
| | 25248.28 | 13349.61 | 3.2 | 13068.5 |
| | 27134.38 | 12608.97 | 3.2 | 13399.9 |
| | | 12787 | | 13078.37 |
| | | 12402.92 | | 12764.63 |
| | | | | 11.44 |
| | | | | 49.04 |
| IL-10 | 2542.02 | 1199.03 | 3749.2 | 59.61 |
| | 2141.67 | 1142.74 | 14565.93 | 117.08 |
| | 5137.94 | 762.56 | 9010.46 | 1019.57 |
| | 2762.07 | 825.18 | 6700 | 807.85 |
| | 6272.75 | 2219.96 | 7800 | 1645.45 |
| | 7879.45 | 1300.24 | 16241.6 | 1457.05 |
| | 5769.33 | 1234.32 | 8482.53 | 1294.99 |
| | 4786.65 | 0.44 | 12679 | 9.87 |
| | 12939.3 | 39.47 | 10000 | 522.68 |
| | 8081.8 | | 9742.86 | |
| | 5839.8 | | 10000 | |
| IL-1α | 88.74 | 476.17 | 60.49 | 60.88 |
| | 122.39 | 180.17 | 121.58 | 82.75 |
| | 129.58 | 690.89 | 150.68 | 326.13 |
| | 85.71 | 316.23 | 10.94 | 239.62 |
| | 123.19 | 152.62 | 10.94 | 69.2 |
| | 591.68 | 169.53 | 472.07 | 169.81 |
| | 708.9 | 45.26 | 541.31 | 119.33 |
| | 835.93 | 21.57 | 142.77 | 8.02 |
| | 707.44 | 31.93 | 444.7 | 25.65 |
| | 339.4 | | 603.38 | |
| IL-12p70 | 715.21 | 166.21 | 69.4 | 18.04 |
| | 102.12 | 106.76 | 70.58 | 32.26 |
| | 270.82 | 244.61 | 12.31 | 186.02 |
| | 295.08 | 3.2 | 24.53 | 139.59 |
| | 262.54 | 107.71 | 29.53 | 37.44 |
| | 57.08 | 70.77 | 10.37 | 143.98 |
| | 22.69 | 207.63 | 3.2 | 83.24 |
| | 31.15 | 21.08 | 3.2 | 3.41 |
| | 4.82 | 21.08 | 4.82 | |
| | 91.86 | | 3.2 | |
| | 14.91 | | | |
| | 26.2 | | | |

These data suggest that PILRβ signaling can augment proinflammatory cytokine production, resulting in increased mortality. These results suggest that the PILR receptors play a critical role in acute inflammatory responses and may be implicated as potential therapeutic targets for septic shock.

D. PILRβ Signaling Increases Mortality and Inflammation During CLP-Induced Peritonitis

Authentic sepsis involves the replication and dissemination of bacteria together with a proinflammatory response. The role of PILRβ during cecal ligation and puncture (CLP)-mediated microbial peritonitis, which closely mimics human sepsis in the clinic, was evaluated. WT and PILRβ−/− mice were subjected to CLP. PILRβ−/− displayed 20% mortality compared to the WT animals, which showed 60% mortality. Since cytokines play a crucial role in the pathogenesis of sepsis, serum cytokine levels of some of the key proinflammatory cytokines that are known to play an important role during sepsis were measured. Prior to CLP, TNFα, IL-10, IL-6 and MCP-1 levels were present at very low levels in WT and PILRβ−/− mice. At 6 h post CLP, however, there was a dramatic increase in the levels of these cytokines and a significant increase was observed for TNFα and MCP-1 in the WT animals compared to the knockouts. An increasing trend in levels of IL-10 among the WT mice was also observed, although the values did not reach significance. IL-6 levels were found to be relatively similar between the two groups at the 6 h time point.

Interestingly, at 24 h post CLP, a considerable reduction in all of the above cytokine levels was observed among the PILRβ−/− mice and the levels were found to be significantly less than the WT levels. After 48 h from the onset of sepsis, serum cytokine levels were found to stabilize and reach almost the levels in naïve mice (see Table 4).

TABLE 4
Serum Cytokine Levels Post CLP

| | WT | | | | PILRβ−/− | | | |
|---|---|---|---|---|---|---|---|---|
| | naïve | 6 h | 24 h | 48 h | naïve | 6 h | 24 h | 48 h |
| IL-6 | 3.2 | 5597.77 | 572.47 | 665.33 | 4.22 | 2669 | 741.41 | 372.24 |
| | 30.67 | 10100 | 1033.74 | 502.12 | 3.2 | 10000 | 516.82 | 361.29 |
| | 0.69 | 10000 | 1049.78 | 406.18 | 3.2 | 10000 | 350.41 | 159.49 |
| | 5.71 | 10000 | 834.63 | 590.11 | 10.14 | 10000 | 460.03 | 392.94 |
| | 3.2 | 10000 | 904.3 | 584.6 | 3.2 | 10000 | 440.04 | 415.12 |
| | | | 1333.755 | 473.14 | | | 870.06 | 418.29 |
| | | | 1276.245 | 567.59 | | | 792.67 | 881.45 |
| | | | 939.455 | 584.73 | | | 856.18 | 297.4 |
| | | | 1333.5 | 584.73 | | | 743.26 | |
| | | | 2164.6 | 1045.55 | | | | |
| | | | 590.28 | | | | | |
| | | | 10000 | | | | | |
| | | | 1940.88 | | | | | |
| | | | 1091.02 | | | | | |
| IL-10 | 3.2 | 797.95 | 126.36 | 19.53 | 3.2 | 1273.72 | 168.26 | 40.29 |
| | 96.03 | 10000 | 626.69 | 16.94 | 3.2 | 11339.1 | 24.6 | 197.4 |
| | 3.2 | 10000 | 263.56 | 33.17 | 3.2 | 6066.65 | 34.37 | 328.29 |
| | 7.68 | 13631.74 | 148.49 | 51.82 | 3.2 | 10000 | 54.65 | 7.35 |
| | 3.2 | 10000 | 412.05 | 23.35 | 3.2 | 1208.46 | 70 | 40.88 |
| | | | 248.25 | 39.97 | | | 133.675 | 3.2 |
| | | | 750 | 3.2 | | | 476.03 | 20.3 |
| | | | 786.37 | 17.35 | | | 900 | 105.75 |
| | | | 479.88 | 17.35 | | | 1092.22 | 421.49 |
| | | | 10752.19 | 481.7 | | | 163.44 | 3.2 |
| | | | 1493.76 | | | | | |
| | | | 301.78 | | | | | |
| | | | 10000 | | | | | |
| TNFα | 4.4 | 145.6 | 7.05 | 4.36 | 5.09 | 13.46 | 6.31 | 7.99 |
| | 17.73 | 205.55 | 11.45 | 6.52 | 5.32 | 87.27 | 7.36 | 13.44 |
| | 4.4 | 71.67 | 10.65 | 11.05 | 4.17 | 102.15 | 6.94 | 7.36 |
| | 5.54 | 136.9 | 11.05 | 7.99 | 7.78 | 118.44 | 13.25 | 5.67 |
| | 7.56 | 131.96 | 12.55 | 8.82 | 5.77 | 14.21 | 9.32 | 5.32 |
| | | | 15.82 | 4.52 | | | 8.45 | 6.67 |
| | | | 12.17 | 5.77 | | | 14.03 | 13.68 |
| | | | 47.4 | 7.34 | | | | 13.25 |
| | | | 112.9 | 7.34 | | | | 4.86 |
| | | | | 11.73 | | | | |
| MCP-1 | 37.53 | 12650.23 | 266.41 | 68.8 | 50.34 | 1165.23 | 943.76 | 88.48 |
| | 56.8 | 5719.63 | 434.9 | 192.9 | 45.13 | 6182.73 | 195.45 | 267.7 |
| | 31.12 | 11561.26 | 353.67 | 202.02 | 17.9 | 7212.36 | 148.95 | 336.44 |
| | 48.65 | 10993.32 | 497.27 | 170.3 | 55.23 | 1754.58 | 252.48 | 68.8 |
| | 17.9 | | 333.11 | 115.75 | 22.7 | 2782.18 | 178.625 | 227.6 |
| | | | 312.715 | 174.33 | | | 357.24 | 52.01 |
| | | | 429.91 | 112.77 | | | 345.95 | 147.11 |
| | | | 521.885 | 96.3 | | | 1042.26 | 133.4 |
| | | | 605.39 | 96.3 | | | 207.54 | 102.87 |
| | | | 732.71 | 464.46 | | | | 35.48 |
| | | | 5610.92 | | | | | |
| | | | 403.45 | | | | | |
| | | | 489.54 | | | | | |
| | | | 9416.02 | | | | | |

E. Lack of PILRβ does not Alter Recruitment of Cells to the Peritoneum or Bacterial Killing

Owing to the engagement of TLRs by microbial products, neutrophils and macrophages are known to have crucial functions as the first line of host defense in the early phase of infection. However, an inadequate recruitment and activation of these myeloid cells could result in severe collateral damage of the adjacent tissue. To determine if the protective phenotype after LPS shock in PILRβ knockout mice could be attributed to recruitment of cells to the peritoneum, WT and PILRβ−/− mice were subjected, as described above, to three different routes of peritonitis: (i) CLP-induced peritonitis, (ii) LPS-induced endotoxic shock and (iii) sterile peritonitis using thioglycollate. Peritoneal cells were harvested at 24 h post CLP, 4-6 h post LPS and 72 h post thioglycollate administration by peritoneal lavage. After counting the cells, no apparent difference in the viable peritoneal infiltrate counts was observed between WT and PILRβ−/− from any of the above mentioned routes (see Table 5).

TABLE 5
Peritoneal Infiltrate after Initiation of Sepsis

| | WT | PILRβ−/− |
|---|---|---|
| 24 h post CLP (cells/ml × 10⁶) | 1.47 | 0.63 |
| | 0.67 | 1.16 |
| | 1.22 | 1.58 |
| | 0.3 | 0.3 |
| | 0.5 | 3 |
| | 0.47 | 0.5 |
| | 0.8 | 0.43 |
| | 0.5 | 0.6 |
| | 0.6 | 0.7 |
| 4 h post LPS (cells/ml × 10⁶) | 1.08 | 1.32 |
| | 1.08 | 1.26 |
| | 1.04 | 2.46 |
| | 0.93 | 1 |
| | 0.7 | 0.45 |
| | 0.79 | 0.62 |
| | 0.82 | 0.52 |
| 72 h post Thioglycollate(cells/ml × 10⁶) | 5.75 | 1.28 |
| | 7.05 | 3.06 |
| | 5.6 | 7.28 |
| | 5.1 | 5.57 |
| | 5.45 | 7.68 |
| | 5.75 | 3.06 |
| | 7.05 | 7.28 |
| | 5.6 | 5.57 |
| | 5.1 | 7.68 |

Furthermore, analysis of surface markers on these peritoneal cells revealed that 40-45% of the cells were found to be positive for CD11b⁺/Ly6⁺G or GR-1⁺ after CLP, 10% post LPS and approximately 25% post thioglycollate treatment. The CD11b⁺/F4/80⁺ macrophage population was found to be anywhere between 45-60% among all modes of sepsis induction. Interestingly, both the granulocyte and macrophage population did not vary significantly between the WT and PILRβ−/− mice (see Table 6).

TABLE 6
Granulocye and Macrophage Infiltration

| | WT | | PILRβ−/− | |
|---|---|---|---|---|
| | Granulocyte | Macrophage | Granulocyte | Macrophage |
| 24 h post CLP | 47.1 | 39 | 33 | 39 |
| (cells/ml × 10⁶) | 53 | 56 | 33.8 | 19 |
| | 37.8 | 32 | 40 | 41 |
| | 45 | 20 | 28 | 45 |
| 4 h post LPS | 9.82 | 5 | 31 | 43 |
| (cells/ml × 10⁶) | 2.79 | 3.4 | 22 | 27 |
| | 11.54 | 2.69 | 28 | 28 |
| | 5 | 13 | 11.5 | 35.8 |
| 72 h post | 27.6 | 18.75 | 57.53 | 40.8 |
| Thioglycollate | 14.54 | 15.38 | 44.46 | 39.23 |
| (cells/ml × 10⁶) | 21.51 | 18.21 | 46.23 | 51.36 |
| | 15.06 | 2.88 | 52.05 | 51.06 |
| | | 22.86 | | 43.26 |
| | 20.22 | 40 | 67.56 | 35 |
| | 27.98 | 23.54 | 57.52 | 57.71 |
| | 25.65 | 26 | 60.46 | 56.7 |
| | 19 | 22.9 | 59 | 56.9 |

In order to determine the role for PILRβ in mediating the bacterial load during sepsis, the bacterial colony numbers in the peritoneum as well as the blood were measured. The results demonstrate that deletion of the PILRβ gene did not have any effect on the bacterial load in the blood or peritoneum at 24 h post CLP (see Table 7).

TABLE 7
Bacterial Burden in Blood and Peritoneum

| | Blood | | Peritoneum | |
|---|---|---|---|---|
| | WT | PILRβ−/− | WT | PILRβ−/− |
| 24 h post CLP | 6000000 | 4.60E+07 | 1.00E+09 | 1000 |
| (cells/ml × 10⁶) | 8000000 | 2400000 | 5000000 | 1.70E+08 |
| | 10000 | 10000000 | 1500000 | 2.50E+09 |
| | 500000 | 200 | 2.00E+08 | 460000 |
| | 10000000 | 0 | | 0 |

F. Altered Expression of PILRα, PILRβ and TLR4 in Peritoneal Macrophages Post LPS-Mediated Endotoxic Shock

Peritoneal cells were isolated 4-6 h post LPS or PBS treatment, counted and processed for staining with primary antibodies against PILRα, anti-PILRβ receptors, and anti-hIL-4 (rIgG1; isotype control). A PE-conjugated goat anti-rat secondary antibody was added subsequent to staining with the primary antibody and the cells were visualized by FACS. The expression levels of PILRα were found to be comparable but elevated in cells obtained from the PBS treated WT or PILRβ−/− mice compared to the cells from LPS-treated WT or PILRβ−/− mice. In contrast, as expected, PILRβ expression was higher in LPS treated WT mice compared to the PBS injected control group.

Both PILRα and β are constitutively expressed by neutrophils and macrophages and in vitro its expression is upregulated in the presence of LPS. However, in cells obtained from WT mice injected with LPS, a synergistic upregulation in expression of only PILRβ was observed, while conversely a marked decrease in PILRα levels was observed in the same mice.

Since TLR4 is the primary receptor for LPS and is a key determinant of LPS responsiveness, expression levels in peritoneal cells from LPS and PBS treated WT and PILRβ−/− mice were evaluated. Even though deletion of PILRβ did not affect levels of TLR4 expression between control WT and knockout mice, a moderate decrease in TLR4 expression levels in the PILRβ−/− mice was observed upon LPS administration (see Table 8).

TABLE 8
TLR4 Expression after LPS Treatment

| | |
|---|---|
| WT | PILRβ−/− |

| PBS | LPS | PBS | LPS |
|---|---|---|---|
| 37 | 59 | 10 | 70 |
| 20 | 60 | 18 | 50 |
| 20 | 28 | 20 | 43 |

The results above, demonstrate that upon LPS injection WT mice succumb quickly to the proinflammatory response, which has been attributed to the high levels of TNFα being produced in circulation shortly after LPS administration.

### III. Generation of PILR Antibodies

Any suitable method for generating monoclonal antibodies may be used. For example, a recipient may be immunized with PILR or a fragment thereof. Any suitable method of immunization can be used. Such methods can include adjuvants, other immunostimulants, repeated booster immunizations, and the use of one or more immunization routes. Any suitable source of PILR can be used as the immunogen for the generation of the non-human antibody of the compositions and methods disclosed herein. Such forms include, but are not limited whole protein, peptide(s), and epitopes generated through recombinant, synthetic, chemical or enzymatic degradation means known in the art. In preferred embodiments the immunogen comprises the extracellular portion of PILR.

Any form of the antigen can be used to generate the antibody that is sufficient to generate a biologically active antibody. Thus, the eliciting antigen may be a single epitope, multiple epitopes, or the entire protein alone or in combination with one or more immunogenicity enhancing agents known in the art. The eliciting antigen may be an isolated full-length protein, a cell surface protein (e.g., immunizing with cells transfected with at least a portion of the antigen), or a soluble protein (e.g., immunizing with only the extracellular domain portion of the protein). The antigen may be produced in a genetically modified cell. The DNA encoding the antigen may genomic or non-genomic (e.g., cDNA) and encodes at least a portion of the extracellular domain. As used herein, the term “portion” refers to the minimal number of amino acids or nucleic acids, as appropriate, to constitute an immunogenic epitope of the antigen of interest. Any genetic vectors suitable for transformation of the cells of interest may be employed, including but not limited to adenoviral vectors, plasmids, and non-viral vectors, such as cationic lipids.

Any suitable method can be used to elicit an antibody with the desired biologic properties to modulate PILR signaling. It is desirable to prepare monoclonal antibodies (mAbs) from various mammalian hosts, such as mice, rats, other rodents, humans, other primates, etc. Description of techniques for preparing such monoclonal antibodies may be found in, e.g., Stites et al. (eds.) BASIC AND CLINICAL IMMUNOLOGY (4th ed.) Lange Medical Publications, Los Altos, Calif., and references cited therein; Harlow and Lane (1988) ANTIBODIES: A LABORATORY MANUAL CSH Press; Goding (1986) MONOCLONAL ANTIBODIES: PRINCIPLES AND PRACTICE (2d ed.) Academic Press, New York, N.Y. Thus, monoclonal antibodies may be obtained by a variety of techniques familiar to researchers skilled in the art. Typically, spleen cells from an animal immunized with a desired antigen are immortalized, commonly by fusion with a myeloma cell. See Kohler and Milstein (1976) Eur. J. Immunol. 6:511-519. Alternative methods of immortalization include transformation with Epstein Barr Virus, oncogenes, or retroviruses, or other methods known in the art. See, e.g., Doyle et al. (eds. 1994 and periodic supplements) CELL AND TISSUE CULTURE: LABORATORY PROCEDURES, John Wiley and Sons, New York, N.Y. Colonies arising from single immortalized cells are screened for production of antibodies of the desired specificity and affinity for the antigen, and yield of the monoclonal antibodies produced by such cells may be enhanced by various techniques, including injection into the peritoneal cavity of a vertebrate host. Alternatively, one may isolate DNA sequences that encode a monoclonal antibody or a antigen binding fragment thereof by screening a DNA library from human B cells according, e.g., to the general protocol outlined by Huse et al. (1989) Science 246:1275-1281.

Other suitable techniques involve selection of libraries of antibodies in phage or similar vectors. See, e.g., Huse et al. supra; and Ward et al. (1989) Nature 341:544-546. The polypeptides and antibodies of the present invention may be used with or without modification, including chimeric or humanized antibodies. Frequently, the polypeptides and antibodies will be labeled by joining, either covalently or non-covalently, a substance that provides for a detectable signal. A wide variety of labels and conjugation techniques are known and are reported extensively in both the scientific and patent literature. Suitable labels include radionuclides, enzymes, substrates, cofactors, inhibitors, fluorescent moieties, chemiluminescent moieties, magnetic particles, and the like. Patents teaching the use of such labels include U.S. Pat. Nos. 3,817,837; 3,850,752; 3,939,350; 3,996,345; 4,277,437; 4,275,149; and 4,366,241. Also, recombinant immunoglobulins may be produced, see Cabilly U.S. Pat. No. 4,816,567; and Queen et al. (1989) Proc. Nat'l Acad. Sci. USA 86:10029-10033; or made in transgenic mice, see Mendez et al. (1997) Nature Genetics 15:146-156. See also Abgenix and Medarex technologies.

Antibodies or binding compositions against predetermined fragments of PILR can be raised by immunization of animals with conjugates of the polypeptide, fragments, peptides, or epitopes with carrier proteins. Monoclonal antibodies are prepared from cells secreting the desired antibody. These antibodies can be screened for binding to normal or defective PILR. These monoclonal antibodies will usually bind with at least a K_{d} of about 1 μM, more usually at least about 300 nM, 30 nM, 10 nM, 3 nM, 1 nM, 300 pM, 100 pM, 30 pM or better, usually determined by ELISA.

Any suitable non-human antibody can be used as a source for the hypervariable region. Sources for non-human antibodies include, but are not limited to, murine (e.g. Mus musculus), rat (e.g. Rattus norvegicus), Lagomorphs (including rabbits), bovine, and primates. For the most part, humanized antibodies are human immunoglobulins (recipient antibody) in which hypervariable region residues of the recipient are replaced by hypervariable region residues from a non-human species (donor antibody) such as mouse, rat, rabbit or non-human primate having the desired specificity, affinity, and capacity. In some instances, Fv framework region (FR) residues of the human immunoglobulin are replaced by corresponding non-human residues. Furthermore, humanized antibodies may comprise residues that are not found in the recipient antibody or in the donor antibody. These modifications are made to further refine antibody performance of the desired biological activity. For further details, see Jones et al. (1986) Nature 321:522-525; Reichmann et al. (1988) Nature 332:323-329; and Presta (1992) Curr. Op. Struct. Biol. 2:593-596.

Anti-PILR antibodies of the present invention may be screened to ensure that they are specific for only one of PILRα and PILRβ as follows. Clearly, anti-PILRα antibodies are raised using immunogen comprising PILRα, or an immunogenic fragment thereof, and anti-PILRβ antibodies are raised using immunogen comprising PILRβ, or an immunogenic fragment thereof. To confirm that the resulting anti-PILR antibodies do not cross-react with the other form of PILR, a competition ELISA may be used. Briefly, the immunogen used to raise the antibody is bound to a well on a plate. Candidate antibodies are added to the wells either alone, or in the presence of varying concentrations of PILRα and PILRβ or fragments thereof. The ratio of PILRα to PILRβ necessary to achieve a given level of inhibition of binding (e.g. 50% reduction) reflects the PILRα-specificity of the candidate antibody. In the case of antibodies raised against PILRβ, or an antigenic fragment thereof, the ratio can more conveniently be expressed as the PILRβ-specificity (the ratio of PILRβ to PILRα). Non-cross-reactive anti-PILR antibodies may exhibit PILRα- or PILRβ-specificities of about two, five, ten, 30, 100, 300, 1000 or more.

Note that it is not necessarily essential that an anti-PILR antibody be non-cross-reactive with the other form of PILR, provided that the antibody nonetheless provides therapeutic benefit. For example, a bispecific agonist antibody against both PILRα and PILRβ may give results similar to those seen with an agonist of PILRα alone, and thus may be therapeutically beneficial. Accordingly, a PILRα agonist need not necessarily be completely non-cross-reactive with PILRβ to show beneficial effect.

Anti-PILR antibodies may also be screened to identify antagonists of PILRβ or agonists of PILRα. One screen for PILRβ antagonists is based on use of PILRβ agonists, such as the putative natural ligand CD99 (SEQ ID NOs: 6 and 8) or agonist anti-PILRβ antibodies (e.g. DX266), to induce degranulation of mast cells. See Example 9. Accordingly, antagonists of PILRβ can be identified by screening for agents (e.g. antibodies) that block this agonist-induced degranulation.

Similarly, agonists of the inhibitory PILRα receptor can be identified based on their ability to suppress mast cell degranulation, for example degranulation induced by agonists of the activating receptor PILRβ or agonists of other activating receptors, such as CD200RL1. See Example 9.

Bispecific antibodies are also useful in the present methods and compositions. As used herein, the term “bispecific antibody” refers to an antibody, typically a monoclonal antibody, having binding specificities for at least two different antigenic epitopes. In one embodiment, the epitopes are from the same antigen. In another embodiment, the epitopes are from two different antigens. Methods for making bispecific antibodies are known in the art. For example, bispecific antibodies can be produced recombinantly using the co-expression of two immunoglobulin heavy chain/light chain pairs. See, e.g., Milstein et al. (1983) Nature 305: 537-39. Alternatively, bispecific antibodies can be prepared using chemical linkage. See, e.g., Brennan et al. (1985) Science 229:81. Bispecific antibodies include bispecific antibody fragments. See, e.g., Holliger et al. (1993) Proc. Natl. Acad. Sci. U.S.A. 90:6444-48, Gruber et al. (1994) J. Immunol. 152:5368.

The parental and engineered forms of the antibodies of the present invention may also be conjugated to a chemical moiety. The chemical moiety may be, inter alia, a polymer, a radionuclide or a cytotoxic factor. Preferably the chemical moiety is a polymer which increases the half-life of the antibody molecule in the body of a subject. Suitable polymers include, but are not limited to, polyethylene glycol (PEG) (e.g., PEG with a molecular weight of 2 kDa, 5 kDa, 10 kDa, 12 kDa, 20 kDa, 30 kDa or 40 kDa), dextran and monomethoxypolyethylene glycol (mPEG). Lee et al., (1999) (Bioconj. Chem. 10:973-981) discloses PEG conjugated single-chain antibodies. Wen et al., (2001) (Bioconj. Chem. 12:545-553) disclose conjugating antibodies with PEG which is attached to a radiometal chelator (diethylenetriaminpentaacetic acid (DTPA)).

The antibodies and antibody fragments may also be conjugated with fluorescent or chemiluminescent labels, including fluorophores such as rare earth chelates, fluorescein and its derivatives, rhodamine and its derivatives, isothiocyanate, phycoerythrin, phycocyanin, allophycocyanin, o-phthaladehyde, fluorescamine, ¹⁵²Eu, dansyl, umbelliferone, luciferin, luminal label, isoluminal label, an aromatic acridinium ester label, an imidazole label, an acridimium salt label, an oxalate ester label, an aequorin label, 2,3-dihydrophthalazinediones, biotin/avidin, spin labels and stable free radicals.

Any method known in the art for conjugating the antibody molecules or protein molecules of the invention to the various moieties may be employed, including those methods described by Hunter et al., (1962) Nature 144:945; David et al., (1974) Biochemistry 13:1014; Pain et al., (1981) J. Immunol. Meth. 40:219; and Nygren, J., (1982) Histochem. and Cytochem. 30:407. Methods for conjugating antibodies and proteins are conventional and well known in the art.

### IV. Nucleic Acid-Based Antagonists of PILRβ

An antagonist of PILRβ also includes nucleic acid-based antagonists that reduce the expression of PILRβ, such as antisense nucleic acids and siRNA. See, e.g., Arenz and Schepers (2003) Naturwissenschaften 90:345-359; Sazani and Kole (2003) J. Clin. Invest. 112:481-486; Pirollo et al. (2003) Pharmacol. Therapeutics 99:55-77; Wang et al. (2003) Antisense Nucl. Acid Drug Devel. 13:169-189. Design of such antagonists is within the skill in the art in light of the known sequence of the mRNA encoding PILRβ, which is available at NCBI Nucleic Acid Sequence Database Accession Numbers NM_{—}013440.3, and is provided herein at SEQ ID NO: 3.

Methods of producing and using siRNA are disclosed, e.g., at U.S. Pat. Nos. 6,506,559 (WO 99/32619); 6,673,611 (WO 99/054459); 7,078,196 (WO 01/75164); 7,071,311 and PCT publications WO 03/70914; WO 03/70918; WO 03/70966; WO 03/74654; WO 04/14312; WO 04/13280; WO 04/13355; WO 04/58940; WO 04/93788; WO 05/19453; WO 05/44981; WO 03/78097 (U.S. patents are listed with related PCT publications). Exemplary methods of using siRNA in gene silencing and therapeutic treatment are disclosed at PCT publications WO 02/096927 (VEGF and VEGF receptor); WO 03/70742 (telomerase); WO 03/70886 (protein tyrosine phosphatase type IVA (Prl3)); WO 03/70888 (Chk1); WO 03/70895 and WO 05/03350 (Alzheimer's disease); WO 03/70983 (protein kinase C alpha); WO 03/72590 (Map kinases); WO 03/72705 (cyclin D); WO 05/45034 (Parkinson's disease). Exemplary experiments relating to therapeutic uses of siRNA have also been disclosed at Zender et al. (2003) Proc. Nat'l. Acad. Sci. (USA) 100:7797; Paddison et al. (2002) Proc. Nat'l. Acad. Sci. (USA) 99:1443; and Sah (2006) Life Sci. 79:1773. siRNA molecules are also being used in clinical trials, e.g., of chronic myeloid leukemia (CML) (ClinicalTrials.gov Identifier: NCT00257647) and age-related macular degeneration (AMD) (ClinicalTrials.gov Identifier: NCT00363714).

Although the term “siRNA” is used herein to refer to molecules used to induce gene silencing via the RNA interference pathway (Fire et al. (1998) Nature 391:806), such siRNA molecules need not be strictly polyribonucleotides, and may instead contain one or more modifications to the nucleic acid to improve its properties as a therapeutic agent. Such agents are occasionally referred to as “siNA” for short interfering nucleic acids. Although such changes may formally move the molecule outside the definition of a “ribo”nucleotide, such molecules are nonetheless referred to as “siRNA” molecules herein. For example, some siRNA duplexes comprise two 19-25 nt (e.g. 21 nt) strands that pair to form a 17-23 basepair (e.g. 19 base pair) polyribonucleotide duplex with TT (deoxyribonucleotide) 3′ overhangs on each strand. Other variants of nucleic acids used to induce gene silencing via the RNA interference pathway include short hairpin RNAs (“shRNA”), for example as disclosed in U.S. Pat. App. Publication No. 2006/0115453.

The sequence of the opposite strand of the siRNA duplexes is simply the reverse complement of the sense strand, with the caveat that both strands have 2 nucleotide 3′ overhangs. That is, for a sense strand “n” nucleotides long, the opposite strand is the reverse complement of residues 1 to (n-2), with 2 additional nucleotides added at the 3′ end to provide an overhang. Where an siRNA sense strand includes two U residues at the 3′ end, the opposite strand also includes two U residues at the 3′ end. Where an siRNA sense strand includes two dT residues at the 3′ end, the opposite strand also includes two dT residues at the 3′ end.

The use of complimentary sequences to arrest translation of mRNAs was described in the late 1970s. See, e.g., Paterson et al. (1977) Proc. Natl. Acad. Sci. (USA) 74:4370-4374; Hastie & Held (1978) Proc. Natl. Acad. Sci. (USA) 75: 1217-1221 and Zamecnik & Stephenson (1978) Proc. Natl. Acad. Sci. (USA) 75:280-284. However, the use of antisense oligonucleotides for selective blockage of specific mRNAs is of recent origin. See, e.g., Weintraub et al. (1985) Trends Genet. 1:22-25 (1985); Loke et al. (1989) Proc. Natl. Acad. Sci. (USA) 86:3474-3478; Mulligan et al. (1993) J. Med. Chem. 36:1923-1937 (1993); and Wagner (1994) Nature 372:333-335. The mechanism of antisense inhibition in cells was previously analyzed and the decrease in mRNA levels mediated by oligonucleotides was shown to be responsible for the decreased expression of several proteins. See Walder & Walder (1988) Proc. Natl. Acad. Sci. (USA) 85:5011-5015; Dolnick (1991) Cancer Invest. 9:185-194; Crooke & LeBleu (1993) Antisense Research and Applications, CRC Press, Inc., Boca Raton, Fla.; Chiang et al. (1991) J. Biol. Chem. 266:18162-18171; and Bennett et al. (1994) J. Immunol. 152:3530-3540. The use of antisense oligonucleotides is recognized as a viable option for the treatment of diseases in animals and man. For example, see U.S. Pat. Nos. 5,098,890; 5,135,917; 5,087,617; 5,166,617; 5,166,195; 5,004,810; 5,194,428; 4,806,463; 5,286,717; 5,276,019; 5,264,423; 4,689,320; 4,999,421 and 5,242,906, which teach the use of antisense oligonucleotides in a variety of diseases including cancer, HIV, herpes simplex virus, influenza virus, HTLV-HI replication, prevention of replication of foreign nucleic acids in cells, antiviral agents specific to CMV, and treatment of latent EBV infections.

An antisense nucleic acid can be provided as an antisense oligonucleotide. See, e.g., Murayama et al. (1997) Antisense Nucleic Acid Drug Dev. 7:109-114. Genes encoding an antisense nucleic acid can also be provided; such genes can be formulated with a delivery enhancing compound and introduced into cells by methods known to those of skill in the art. For example, one can introduce a gene that encodes an antisense nucleic acid in a viral vector, such as, for example, in hepatitis B virus (see, e.g., Ji et al. (1997) J. Viral Hepat. 4:167-173); in adeno-associated virus (see e.g., Xiao et al. (1997) Brain Res. 756:76-83; or in other systems including, but not limited, to an HVJ (Sendai virus)-liposome gene delivery system (see, e.g., Kaneda et al. (1997) Ann. N.Y. Acad. Sci. 811:299-308); a “peptide vector” (see, e.g., Vidal et al. (1997) CR Acad. Sci III 32:279-287); as a gene in an episomal or plasmid vector (see, e.g., Cooper et al. (1997) Proc. Natl. Acad. Sci. (U.S.A.) 94:6450-6455, Yew et al. (1997) Hum Gene Ther. 8:575-584); as a gene in a peptide-DNA aggregate (see, e.g., Niidome et al. (1997) J. Biol. Chem. 272:15307-15312); as “naked DNA” (see, e.g., U.S. Pat. No. 5,580,859 and U.S. Pat. No. 5,589,466); in lipidic vector systems (see, e.g., Lee et al. (1997) Crit. Rev. Ther. Drug Carrier Syst. 14:173-206); polymer coated liposomes (U.S. Pat. Nos. 5,213,804 and 5,013,556); cationic liposomes (U.S. Pat. Nos. 5,283,185; 5,578,475; 5,279,833; 5,334,761); gas filled microspheres (U.S. Pat. No. 5,542,935), ligand-targeted encapsulated macromolecules (U.S. Pat. Nos. 5,108,921; 5,521,291; 5,554,386; and 5,166,320).

### V. Pharmaceutical Compositions

To prepare pharmaceutical or sterile compositions including PILR antibodies, the polypeptide analogue or mutein, antibody thereto, or nucleic acid thereof, is admixed with a pharmaceutically acceptable carrier or excipient. See, e.g., Remington's Pharmaceutical Sciences and U.S. Pharmacopeia: National Formulary, Mack Publishing Company, Easton, Pa. (1984).

Formulations of therapeutic and diagnostic agents may be prepared by mixing with physiologically acceptable carriers, excipients, or stabilizers in the form of, e.g., lyophilized powders, slurries, aqueous solutions or suspensions. See, e.g., Hardman et al. (2001) Goodman and Gilman's The Pharmacological Basis of Therapeutics, McGraw-Hill, New York, N.Y.; Gennaro (2000) Remington: The Science and Practice of Pharmacy, Lippincott, Williams, and Wilkins, New York, N.Y.; Avis et al. (eds.) (1993) Pharmaceutical Dosage Forms Parenteral Medications, Marcel Dekker, NY; Lieberman, et al. (eds.) (1990) Pharmaceutical Dosage Forms: Tablets, Marcel Dekker, NY; Lieberman et al. (eds.) (1990) Pharmaceutical Dosage Forms: Disperse Systems, Marcel Dekker, NY; Weiner and Kotkoskie (2000) Excipient Toxicity and Safety, Marcel Dekker, Inc., New York, N.Y.

Toxicity and therapeutic efficacy of the antibody compositions, administered alone or in combination with an immunosuppressive agent, can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio of LD₅₀ to ED₅₀. Antibodies exhibiting high therapeutic indices are preferred. The data obtained from these cell culture assays and animal studies can be used in formulating a range of dosage for use in human. The dosage of such compounds lies preferably within a range of circulating concentrations that include the ED₅₀ with little or no toxicity. The dosage may vary within this range depending upon the dosage form employed and the route of administration.

The mode of administration is not particularly important. Suitable routes of administration may, for example, include oral, rectal, transmucosal, or intestinal administration; parenteral delivery, including intramuscular, subcutaneous, intramedullary injections, as well as intrathecal, direct intraventricular, intravenous, intraperitoneal, intranasal, or intraocular injections. Administration of antibody used in the pharmaceutical composition or to practice the method of the present invention can be carried out in a variety of conventional ways, such as oral ingestion, inhalation, topical application or cutaneous, subcutaneous, intraperitoneal, parenteral, intraarterial or intravenous injection.

Alternately, one may administer the antibody in a local rather than systemic manner, for example, via injection of the antibody directly into an arthritic joint or pathogen-induced lesion characterized by immunopathology, often in a depot or sustained release formulation. Furthermore, one may administer the antibody in a targeted drug delivery system, for example, in a liposome coated with a tissue-specific antibody, targeting, for example, arthritic joint or pathogen-induced lesion characterized by immunopathology. The liposomes will be targeted to and taken up selectively by the afflicted tissue.

Selecting an administration regimen for a therapeutic depends on several factors, including the serum or tissue turnover rate of the entity, the level of symptoms, the immunogenicity of the entity, and the accessibility of the target cells in the biological matrix. Preferably, an administration regimen maximizes the amount of therapeutic delivered to the patient consistent with an acceptable level of side effects. Accordingly, the amount of biologic delivered depends in part on the particular entity and the severity of the condition being treated. Guidance in selecting appropriate doses of antibodies, cytokines, and small molecules are available. See, e.g., Wawrzynczak (1996) Antibody Therapy, Bios Scientific Pub. Ltd, Oxfordshire, UK; Kresina (ed.) (1991) Monoclonal Antibodies, Cytokines and Arthritis, Marcel Dekker, New York, N.Y.; Bach (ed.) (1993) Monoclonal Antibodies and Peptide Therapy in Autoimmune Diseases, Marcel Dekker, New York, N.Y.; Baert et al. (2003) New Engl. J. Med. 348:601-608; Milgrom et al. (1999) New Engl. J. Med. 341:1966-1973; Slamon et al. (2001) New Engl. J. Med. 344:783-792; Beniaminovitz et al. (2000) New Engl. J. Med. 342:613-619; Ghosh et al. (2003) New Engl. J. Med. 348:24-32; Lipsky et al. (2000) New Engl. J. Med. 343:1594-1602.

Determination of the appropriate dose is made by the clinician, e.g., using parameters or factors known or suspected in the art to affect treatment or predicted to affect treatment. Generally, the dose begins with an amount somewhat less than the optimum dose and it is increased by small increments thereafter until the desired or optimum effect is achieved relative to any negative side effects. Important diagnostic measures include those of symptoms of, e.g., the inflammation or level of inflammatory cytokines produced. Preferably, a biologic that will be used is substantially derived from the same species as the animal targeted for treatment (e.g. a humanized antibody for treatment of human subjects), thereby minimizing any immune response to the reagent.

Antibodies, antibody fragments, and cytokines can be provided by continuous infusion, or by doses at intervals of, e.g., one day, 1-7 times per week, one week, two weeks, monthly, bimonthly, etc. Doses may be provided intravenously, subcutaneously, topically, orally, nasally, rectally, intramuscular, intracerebrally, intraspinally, or by inhalation. A preferred dose protocol is one involving the maximal dose or dose frequency that avoids significant undesirable side effects. A total weekly dose is generally at least 0.05 μg/kg, 0.2 μg/kg, 0.5 μg/kg, 1 μg/kg, 10 μg/kg, 100 μg/kg, 0.2 mg/kg, 1.0 mg/kg, 2.0 mg/kg, 10 mg/kg, 25 mg/kg, 50 mg/kg body weight or more. See, e.g., Yang et al. (2003) New Engl. J. Med. 349:427-434; Herold et al. (2002) New Engl. J. Med. 346:1692-1698; Liu et al. (1999) J. Neurol. Neurosurg. Psych. 67:451-456; Portielji et al. (20003) Cancer Immunol. Immunother. 52:133-144. The desired dose of a small molecule therapeutic, e.g., a peptide mimetic, natural product, or organic chemical, is about the same as for an antibody or polypeptide, on a moles/kg basis.

As used herein, “inhibit” or “treat” or “treatment” includes a postponement of development of the symptoms associated with a microbial infection and/or a reduction in the severity of such symptoms that will or are expected to develop. Thus, the terms denote that a beneficial result has been conferred on a vertebrate subject with an microbial infection, or with the potential to develop such a disease or symptom.

As used herein, the term “therapeutically effective amount” or “effective amount” refers to an amount of an PILR-specific binding compound, e.g. and antibody, that when administered alone or in combination with an additional therapeutic agent to a cell, tissue, or subject is effective to prevent or ameliorate the autoimmune disease or pathogen-induced immunopathology associated disease or condition or the progression of the disease. A therapeutically effective dose further refers to that amount of the compound sufficient to result in amelioration of symptoms, e.g., treatment, healing, prevention or amelioration of the relevant medical condition, or an increase in rate of treatment, healing, prevention or amelioration of such conditions. When applied to an individual active ingredient administered alone, a therapeutically effective dose refers to that ingredient alone. When applied to a combination, a therapeutically effective dose refers to combined amounts of the active ingredients that result in the therapeutic effect, whether administered in combination, serially or simultaneously. An effective amount of therapeutic will decrease the symptoms typically by at least 10%; usually by at least 20%; preferably at least about 30%; more preferably at least 40%, and most preferably by at least 50%.

Methods for co-administration or treatment with a second therapeutic agent, e.g., a cytokine, antibody, steroid, chemotherapeutic agent, antibiotic, or radiation, are well known in the art, see, e.g., Hardman et al. (eds.) (2001) Goodman and Gilman's The Pharmacological Basis of Therapeutics, 10th ed., McGraw-Hill, New York, N.Y.; Poole and Peterson (eds.) (2001) Pharmacotherapeutics for Advanced Practice: A Practical Approach, Lippincott, Williams & Wilkins, Phila., PA; Chabner and Longo (eds.) (2001) Cancer Chemotherapy and Biotherapy, Lippincott, Williams & Wilkins, Phila., PA. Antibiotics can include known antibacterial, anti-fungal, and anti-viral agents. Antibacterial agents can include, but are not limited to beta lactam agents that inhibit of cell wall synthesis, such as penicillins, cephalosporins, cephamycins, carbopenems, monobactam; and non beta lactam agents that inhibit cell wall synthesis, such as vancomycin and teicoplanin. Other antibiotics can inhibit cellular activity such as protein and nucleic acid synthesis. These agents include, but are not limited to, macrolides, tetracyclines, aminoglycosides, chloramphenicol, sodium fusidate, sulphonamides, quinolones, and azoles.

Known anti-fungals include, but are not limited to, allylamines and other non-azole ergosterol biosynthesis inhibitors, such as terbinafine; antimetabolites, such as flucytosine; azoles, such as fluconazole, itraconazole, ketoconazole, ravuconazole, posaconazole, and voriconazole; glucan synthesis inhibitors, such as caspofungin, micafungin, and anidulafungin; polyenes, such as amphotericin B, amphotericin B Lipid Complex (ABLC), amphotericin B colloidal dispersion (ABCD), liposomal amphotericin B (L-AMB), and liposomal nystatin; and other systemic agents, such as griseofulvin.

Anti-virals include any drug that destroys viruses. Antivirals may include interferons which function to inhibits replication of the virus, protease inhibitors, and reverse transcriptase inhibitors.

Typical veterinary, experimental, or research subjects include monkeys, dogs, cats, rats, mice, rabbits, guinea pigs, horses, and humans.

### VI. Uses

The present invention provides methods for using anti-PILR antibodies and fragments thereof for the treatment and diagnosis of inflammatory disorders resulting from microbial infections, e.g., sepsis.

The present invention provides methods for diagnosing the presence of or propensity to develop sepsis by analyzing expression levels of PILR in test cells, tissue or bodily fluids compared with PILR levels in cells, tissues or bodily fluids of preferably the same type from a control. As demonstrated herein, an increase in level of PILR expression, for example, in the patient versus the control is associated with the presence of cancer or microbial infection.

Typically, for a quantitative diagnostic assay, a positive result indicating the patient tested has cancer or an infectious disease, is one in which the cells, tissues, or bodily fluids has an PILR expression level at least two times higher, five times higher, ten times higher, fifteen times higher, twenty times higher, twenty-five times higher.

Assay techniques that may be used to determine levels of gene and protein expression, such as PILR, of the present inventions, in a sample derived from a host are well known to those of skill in the art. Such assay methods include radioimmunoassays, reverse transcriptase PCR (RT-PCR) assays, quantitative real-time PCR assays, immunohistochemistry assays, in situ hybridization assays, competitive-binding assays, western blot assays, ELISA assays, and flow cytometric assays, for example, two color FACS analysis for M2 versus M1 phenotyping of tumor-associated macrophages (Mantovani et al., (2002) TRENDS in Immunology 23:549-555).

An ELISA assay initially comprises preparing an antibody specific to PILR. In addition, a reporter antibody generally is prepared that binds specifically to PILR. The reporter antibody is attached to a detectable reagent such as radioactive, fluorescent or an enzymatic reagent, for example horseradish peroxidase enzyme or alkaline phosphatase.

To carry out the ELISA, at least one of the PILR-specific antibody is incubated on a solid support, e.g., a polystyrene dish that binds the antibody. Any free protein binding sites on the dish are then covered by incubating with a non-specific protein, such as bovine serum albumin. Next, the sample to be analyzed is incubated in the dish, during which time PILR binds to the specific PILR antibody attached to the polystyrene dish. Unbound sample is washed out with buffer. A reporter antibody specifically directed to PILR and linked to horseradish peroxidase is placed in the dish resulting in binding of the reporter antibody to any monoclonal antibody bound to PILR. Unattached reporter antibody is then washed out. Reagents for peroxidase activity, including a colorimetric substrate are then added to the dish. Immobilized peroxidase, linked to PILR antibodies, produces a colored reaction product. The amount of color developed in a given time period is proportional to the amount of PILR protein present in the sample. Quantitative results typically are obtained by reference to a standard curve.

A competition assay may be employed wherein antibodies specific to PILR are attached to a solid support and labeled PILR and a sample derived from the host are passed over the solid support and the amount of label detected attached to the solid support can be correlated to a quantity of PILR in the sample.

The above tests may be carried out on samples derived from a variety of cells, bodily fluids and/or tissue extracts such as homogenates or solubilized tissue obtained from a patient. Tissue extracts are obtained routinely from tissue biopsy and autopsy material. Bodily fluids useful in the present invention include blood, urine, saliva or any other bodily secretion or derivative thereof. The term “blood” is meant to include whole blood, plasma, serum or any derivative of blood.

The broad scope of this invention is best understood with reference to the following examples, which are not intended to limit the inventions to the specific embodiments. The specific embodiments described herein are offered by way of example only, and the invention is to be limited by the terms of the appended claims, along with the full scope of equivalents to which such claims are entitled.

### EXAMPLES

### Example 1

### General Methods

Standard methods in molecular biology are described. Maniatis et al. (1982) Molecular Cloning, A Laboratory Manual, Cold Spring Harbor Laboratory Press, Cold Spring Harbor, N.Y.; Sambrook and Russell (2001) Molecular Cloning, 3^{rd} ed., Cold Spring Harbor Laboratory Press, Cold Spring Harbor, N.Y.; Wu (1993) Recombinant DNA, Vol. 217, Academic Press, San Diego, Calif. Standard methods also appear in Ausbel et al. (2001) Current Protocols in Molecular Biology, Vols. 1-4, John Wiley and Sons, Inc. New York, N.Y., which describes cloning in bacterial cells and DNA mutagenesis (Vol. 1), cloning in mammalian cells and yeast (Vol. 2), glycoconjugates and protein expression (Vol. 3), and bioinformatics (Vol. 4).

Methods for protein purification including immunoprecipitation, chromatography, electrophoresis, centrifugation, and crystallization are described. Coligan et al. (2000) Current Protocols in Protein Science, Vol. 1, John Wiley and Sons, Inc., New York. Chemical analysis, chemical modification, post-translational modification, production of fusion proteins, glycosylation of proteins are described. See, e.g., Coligan et al. (2000) Current Protocols in Protein Science, Vol. 2, John Wiley and Sons, Inc., New York; Ausubel et al. (2001) Current Protocols in Molecular Biology, Vol. 3, John Wiley and Sons, Inc., NY, N.Y., pp. 16.0.5-16.22.17; Sigma-Aldrich, Co. (2001) Products for Life Science Research, St. Louis, Mo.; pp. 45-89; Amersham Pharmacia Biotech (2001) BioDirectory, Piscataway, N.J., pp. 384-391. Production, purification, and fragmentation of polyclonal and monoclonal antibodies are described. Coligan et al. (2001) Current Protocols in Immunology, Vol. 1, John Wiley and Sons, Inc., New York; Harlow and Lane (1999) Using Antibodies, Cold Spring Harbor Laboratory Press, Cold Spring Harbor, N.Y.; Harlow and Lane, supra. Standard techniques for characterizing ligand/receptor interactions are available. See, e.g., Coligan et al. (2001) Current Protocols in Immunology, Vol. 4, John Wiley, Inc., New York.

Methods for flow cytometry, including fluorescence activated cell sorting detection systems (FACS®), are available. See, e.g., Owens et al. (1994) Flow Cytometry Principles for Clinical Laboratory Practice, John Wiley and Sons, Hoboken, N.J.; Givan (2001) Flow Cytometry, 2^{nd} ed.; Wiley-Liss, Hoboken, N.J.; Shapiro (2003) Practical Flow Cytometry, John Wiley and Sons, Hoboken, N.J. Fluorescent reagents suitable for modifying nucleic acids, including nucleic acid primers and probes, polypeptides, and antibodies, for use, e.g., as diagnostic reagents, are available. Molecular Probes (2003) Catalogue, Molecular Probes, Inc., Eugene, Oreg.; Sigma-Aldrich (2003) Catalogue, St. Louis, Mo.

Standard methods of histology of the immune system are described. See, e.g., Muller-Harmelink (ed.) (1986) Human Thymus: Histopathology and Pathology, Springer Verlag, New York, N.Y.; Hiatt, et al. (2000) Color Atlas of Histology, Lippincott, Williams, and Wilkins, Phila, Pa.; Louis, et al. (2002) Basic Histology: Text and Atlas, McGraw-Hill, New York, N.Y.

Software packages and databases for determining, e.g., antigenic fragments, leader sequences, protein folding, functional domains, glycosylation sites, and sequence alignments, are available. See, e.g., GenBank, Vector NTI® Suite (Informax, Inc, Bethesda, Md.); GCG Wisconsin Package (Accelrys, Inc., San Diego, Calif.); DeCypher® (TimeLogic Corp., Crystal Bay, Nev.); Menne et al. (2000) Bioinformatics 16: 741-742; Menne et al. (2000) Bioinformatics Applications Note 16:741-742; Wren et al. (2002) Comput. Methods Programs Biomed. 68:177-181; von Heijne (1983) Eur. J. Biochem. 133:17-21; von Heijne (1986) Nucleic Acids Res. 14:4683-4690.

### Example 2

### Reagents and Antibodies

Agonist antibodies against the activating PILRβ and inhibitory PILRα for both human and mouse were generated in-house as described in Fournier et al. (2000) J. Immunol. 165:1197-209. Briefly, female Lewis rats were immunized at regular intervals with a fusion protein consisting of the extracellular domain of mouse or human PILRα/β gene fused to the Fc domain of hIg as described in Wright, et al. (2003) J. Immunol. 171:3034-3046. Hybridomas were initially selected that recognized PILRα/β-Ig (but not the control Ig) fusion protein in indirect ELISA. Hybridomas were then further selected based on their ability to recognize neutrophils, PBMCs and appropriate stably transfected mast cell lines.

### Example 3

### Mice

C57BL/6J mice were purchased from Jackson Laboratories, Sacramento, Calif. PILRβ−/− mice were obtained from Xenogen. Briefly, the entire coding region (exons 1 through 5) was removed in the wildtype allele and replaced with the neomycin cassette to obtain the recombinant allele. PILRβ−/− were generated on a C57BL/6J background. The resulting mice were tested for the absence of the PILRβ gene by analyzing their genetic background by simple sequence length polymorphism.

### Example 4

### Cell Isolation

Human neutrophils and PBMCs were purified from the peripheral blood of healthy donors by dextran sedimentation, hypotonic lysis of RBCs and centrifugation through Ficoll Hypague as described previously [24]. CD14⁺ monocytes were further purified from PBMCs by magnetic bead sorting using CD14 MicroBeads (Miltenyi, Bergisch Gladbach, Germany). For mouse cells, whole blood was obtained from 6-8 wk old mice by cardiac puncture and mixed with five times the volume of lysis buffer (44.5 g ammonium chloride, 5.0 g potassium bicarbonate, 2 mM EDTA, pH 7.3) for 5 minutes to remove the RBCs. The mixture was spun down and the pellet containing the leukocytes was resuspended in an appropriate volume of PBS.

### Example 5

### Cell Staining and Stimulation

Purified neutrophils, monocytes and monocyte derived DCs and macrophages were incubated with goat IgG for 30 minutes at 4 degC to block Fc receptors. Cells were further incubated for 1 h at 4° C. with primary antibodies (anti-PILRβ, anti-PILRα or anti-PILRα/β), followed by an incubation step with PE-conjugated goat anti-rat secondary antibody for an additional 30 minutes. Cells were washed and analyzed by FACS. Neutrophils stimulated with media or LPS (10 ng/ml) for 16 h at 37 deg C. were stained with the different PILRα/β mAbs as described above, and surface expression of PILRα/β was analyzed by flow cytometry.

### Example 6

### Endotoxic Shock

Female C57BL/6J mice, 6-8 wk old were subjected to s.c. doses (600 ug/mouse-1 mg/mouse) of agonistic anti-mPILRβ and PILRα. At 24 h post antibody administration, the mice were induced for LPS-mediated endotoxemia by injecting 150-200 ug of LPS (Salmonella typhimurium, Alexis Biochemicals, San Diego, Calif.) i.p. LPS was also injected in a similar manner in PILRβ−/− mice and their corresponding age matched wt controls in order to determine the in vivo effects of endotoxic shock in these mice. In both cases mice were monitored for 1 week for survival or for failure to upright themselves, at which point they were euthanized. At 1 h post LPS administration a sample of blood was taken and plasma TNFα was determined by ELISA and levels of other circulating cytokines was measured using Luminex® detection assay (Millipore).

### Example 7

### Peritoneal Cell Isolation and Characterization

Peritoneal cells from PILRβ−/− and wt mice were isolated by peritoneal lavage as described in Turnbull, et al. (2005) J. Exp. Med. 202:363-369. Briefly at 4-6 h post i.p. administration of LPS, cells were harvested with 3×3 ml of RPMI 1640 containing 10% FCS, 2 mM Glutamine, Penicillin-Streptomycin, 1 mM Na-pyruvate, 100 ug/ml of nonessential amino acids and 2 mM EDTA. Sterile peritonitis was induced by i.p. injection of thioglycollate media and cells were harvested 48-72 h post insult. Cells were washed and the total number of peritoneal cells in both cases was determined using a Vi-cell counter (BD Biosciences). Cell differential in the peritoneal fluid was determined by flow cytometry. Peritoneal cells were stained with PE-conjugated anti-F4/80, FITC-conjugated anti-CD11b and APC-conjugated anti-Ly6G for 45 minutes on ice. Cells were washed twice with FACs buffer and acquired on a flow cytometer (FACScalibur™, BD Biosciences).

To evaluate any differences in cell surface expression of the PILRα/β and TLR4 receptors, peritoneal cells from wt and PILRβ−/− mice were harvested 4 h post LPS injection. The cells were incubated with goat IgG (1 ug/10⁶ cells) for 20 minutes at 4° C. to block the Fc receptors. The cells were incubated with anti-PILRβ mAb, anti-PILRα mAb, PE-conjugated TLR4 mAb and isotype specific antibody rIgG1a for 1 h at 4° C. Cells were washed and after a further incubation step with PE-conjugated goat anti-rat secondary antibody, the cells were analyzed by FACS.

### Example 8

### Cecal Ligation and Puncture (CLP)

Polymicrobial sepsis was induced by cecal ligation and puncture as previously described in Oberholzer, et al. (2001) Proc. Natl. Acad. Sci. USA 98:11503-11508; Scumpia, et al. (2005) J. Immunol. 175:3282-3286; and Delano, et al. (2007) J. Exp. Med. 204:1463-1474. Mice were anesthetized with isofluorane and the cecum was exteriorized through an abdominal incision. The cecum was then ligated distal to the ileocecal valve and punctured through-and-through with a 19G needle. The cecum was replaced and the abdominal incision was closed in a single layer with sterile surgical clips. Mice were then injected s.c. with 800 ul of normal saline and laid in the supine position in their respective cages. The mice were monitored for survival over two weeks. At the times indicated, a sample of blood was taken and the serum cytokine levels were measured using Luminex® detection assay. The bacterial load in the blood was measured by making a 1:1 dilution of 30 ul of blood with 30 ul of heparin solution. The samples were further serially diluted 1:10 in sterile PBS and subsequently plated on sheep's blood agar plates. Colonies were counted after overnight incubation at 37 deg C.

### Example 9

### Generation and Characterization of Anti-FDF03/PILR Antibodies

Agonist antibodies against the activating PILRβ and inhibitory PILRα for both human and mouse were generated in-house as described previously (see, e.g., Fournier, et al. supra). Briefly, female Lewis rats were immunized at regular intervals with a fusion protein consisting of the extracellular domain of mouse or human PILRα/β gene fused to the Fc domain of hIg as described previously (Wright et al. (2003) J. Immunol. 171:3034-3046). Hybridomas were initially selected that recognized PILRα/β-Ig (but not the control Ig) fusion protein in indirect ELISA. Hybridomas were then further selected based on their ability to recognize neutrophils, PBMCs and appropriate stably transfected mast cell lines.

Antibodies were further characterized as agonist antibodies specific for murine PILRα (DX276) or PILRβ (DX266) based on their ability to inhibit or activate degranulation (measured by β-hexosaminidase release) in mast cell transfectants expressing PILRα (e.g. DT866) or expressing PILRβ (e.g. DT865), respectively. See Zhang et al. (2004) 173:6786 and Cherwinski et al. (2005) J. Immunol. 174:1348, both of which are hereby incorporated by reference. Briefly, to determine whether an antibody is a PILRβ agonist, degranulation is triggered by incubating 1×10⁶ mouse mast cells with the potential PILRβ agonist antibody for one hour in RPMI 1640 medium in 96-well plates.

To determine whether an antibody was a mouse PILRα agonist, degranulation was triggered by incubating 1×10⁶ mouse mast cells with an agonist antibody that binds to the activating receptor CD200RLa (DX89) for one hour in RPMI 1640 medium in 96-well plates, in the presence and in the absence of the potential PILRα agonist antibody.

For both PILRβ and PILRα agonist assays, a 20 μl sample of supernatant was then mixed with 60 μl of the β-hexosaminidase substrate p-nitrophenol-N-acetyl-β-D-glucosaminide (Sigma-Aldrich, St. Louis, Mo., USA) at 1.3 mg/ml in 0.1 M citric acid, pH 4.5. After 3-4 hours at 37° C., 100 μl of stop solution (0.2 M glycine, 0.2 M NaCl, pH 10.7) was added, and the OD₄₀₅₋₆₅₀ was read using a microplate reader (Molecular Devices, Sunnyvale, Calif., USA). Higher OD₄₀₅₋₆₅₀ reflects more β-hexosaminidase in the supernatant, which in turn reflects enhanced degranulation of the mast cells being assayed. See also U.S. Pat. App. Pub. No. 20030223991.

An antibody that specifically binds to mouse PILRβ and triggers degranulation in mast cell transfectants expressing PILRβ (such as DT865), as measured by β-hexosaminidase release, is an agonistic anti-PILRβ antibody. Such data are and particularly reliable if degranulation is triggered in a concentration-dependent manner.

Similarly, an antibody that specifically binds to PILRα and inhibits degranulation in mast cell transfectants expressing PILRα (such as DT866) that are stimulated with DX87 (an antibody specific for the activating receptor CD200RLa), as measured by β-hexosaminidase release, is an agonistic anti-PILRα antibody. See U.S. Pat. App. Pub. No. 20030223991, the disclosure of which is hereby incorporated by reference in its entirety. Such data are and particularly reliable if degranulation is inhibited in a concentration-dependent manner.

To determine whether an antibody is a mouse PILRβ antagonist, degranulation is triggered by incubating 1×10⁶ mouse mast cells with a ligand for PILRβ, such as murine CD99, for one hour in RPMI 1640 medium in 96-well plates, in the presence and in the absence of the potential PILRβ antagonist antibody. An antibody that specifically binds to PILRβ and inhibits degranulation in mast cell transfectants expressing PILRβ (such as DT865) that are stimulated with CD99, as measured by β-hexosaminidase release, is an antagonistic anti-PILRβ antibody. Such data are and particularly reliable if degranulation is inhibited in a concentration-dependent manner.

One of skill in the art would recognize that the screening assays described in this example for the identification of antagonists of mouse PILRβ and agonists of mouse PILRα could be adapted for identification of antagonists of human PILRβ and agonists of human PILRα. Specifically, antibodies raised to human forms of PILRβ and PILRα could be screened in a mast cell degranulation assays involving human (rather than mouse) mast cells. Human cell lines or animals could be engineered to express the human CD200R1L, PILRβ and/or PILRα for use in screening. Human CD200R1L, also known as CD200RLa, is an activating form of CD200R and is further described at Gene ID No. 344807 at the NCBI website, and the nucleic acid and polypeptide sequences are provided at RefSeq NM_{—}001008784.2 and NP_{—}001008784.2, respectively.

For identification of human PILRα agonists, an agonist antibody specific for the activating human receptor CD200R1L may be used to stimulate degranulation, rather than DX87. Alternatively, an agonist antibody for human PILRβ, previously selected for its ability to stimulate mast cell degranulation, may be used in place of DX87 to stimulate degranulation in human mast cells expressing both expressing both PILRβ and PILRα.

For identification of human PILRβ antagonists, human CD99 (SEQ ID NOs: 6 and 8) is used in place of mouse CD99-like molecule to stimulate degranulation. See, e.g., Shiratori et al. (2004) J. Exp. Med. 199:525 at 532.

A listing of sequence identifiers is provides at Table 9.

TABLE 9
Sequence Identifiers

| SEQ | | |
|---|---|---|
| ID NO: | Description | RefSeq |
| 1 | human PILRα nucleic acid | NM_013439.2 |
| 2 | human PILRα polypeptide | NP_038467.2 |
| 3 | human PILRβ nucleic acid | NM_013440.3 |
| 4 | human PILRβ polypeptide | NP_038468.3 |
| 5 | human CD99 (long isoform) nucleic acid | NM_002414.3 |
| 6 | human CD99 (long isoform) polypeptide | NP_002405.1 |
| 7 | human CD99 (short isoform) nucleic | NM_001122898.1 |
| | acid | |
| 8 | human CD99 (short isoform) polypeptide | NP_001116370.1 |

1. A method of treating sepsis comprising administering to a subject in need of such treatment, an effective amount of an antagonist of PILRβ.

2. The method of claim 1 wherein the antagonist of PILRβ is an antibody, antibody fragment, antibody conjugate, a soluble PILRβ polypeptide, or a soluble PILRβ polypeptide fused to a heterologous protein.

3. The method of claim 2, wherein the antibody, antibody fragment, or antibody conjugate comprises:
i) a polyclonal antibody or fragment thereof;
ii) a monoclonal antibody or fragment thereof;
iii) a recombinant antibody or fragment thereof;
iv) a humanized antibody or fragment thereof; or
v) a fully human antibody or fragment thereof.

4. The method of claim 1, wherein the antagonist of PILRβ reduces at least one symptom of sepsis.

5. The method of claim 1, wherein the antagonist of PILRβ is administered with at least one antibiotic having bacteriocidal or bacteriostatic activity.

6. A method of treating sepsis comprising administering to a subject in need of such treatment, an effective amount of an agonist of PILRα.

7. The method of claim 6 wherein the agonist of PILRα is an antibody, antibody fragment, or antibody conjugate.

8. The method of claim 7, wherein the antibody, antibody fragment, or antibody conjugate comprises:
i) a polyclonal antibody or fragment thereof;
ii) a monoclonal antibody or fragment thereof;
iii) a recombinant antibody or fragment thereof;
iv) a humanized antibody or fragment thereof; or
v) a fully human antibody or fragment thereof.

9. The method of claim 6, wherein the agonist of PILRα reduces at least one symptom of sepsis.

10. The method of claim 6, wherein the agonist of PILRα is administered with at least one antibiotic having bateriocidal or bacteriostatic activity.

11. A method of prophylactically treating a subject for sepsis comprising administering to the subject in need of such treatment an effective amount of an antagonist of PILRβ.

12. The method of claim 11 wherein the antagonist of PILRβ is an antibody, antibody fragment, antibody conjugate, a soluble PILRβ polypeptide, or a soluble PILRβ polypeptide fused to a heterologous protein.

13. The method of claim 12, wherein the antibody, antibody fragment, or antibody conjugate comprises:
i) a polyclonal antibody or fragment thereof;
ii) a monoclonal antibody or fragment thereof;
iii) a recombinant antibody or fragment thereof;
iv) a humanized antibody or fragment thereof; or
v) a fully human antibody or fragment thereof.

14. (canceled)

15. The method of claim 11, wherein the antagonist of PILRβ is administered with at least one antibiotic having bacteriocidal or bacteriostatic activity.

16. A method of prophylactically treating a subject against sepsis comprising administering to the subject in need of such treatment an effective amount of an agonist of PILRα.

17. The method of claim 16 wherein the agonist of PILRα is antibody, antibody fragment or antibody conjugate.

18. The method of claim 17, wherein the antibody, antibody fragment, or antibody conjugate comprises:
i) a polyclonal antibody or fragment thereof;
ii) a monoclonal antibody or fragment thereof;
iii) a recombinant antibody or fragment thereof;
iv) a humanized antibody or fragment thereof; or
v) a fully human antibody or fragment thereof.

19. (canceled)

20. The method of claim 16, wherein the agonist of PILRα is administered with at least one antibiotic having bacteriocidal or bacteriostatic activity.

21. The method of claim 1 wherein the antagonist of PILRβ is a nucleic acid antagonist selected from the group consisting of an antisense nucleic acid or an siRNA.

22. The method of claim 11 wherein the antagonist of PILRβ is a nucleic acid antagonist selected from the group consisting of an antisense nucleic acid or an siRNA.
